(19) United States
(12) Patent Application Publication
(10) Pub. No.: US 2024/0141386 A1
(43) Pub. Date: May 2, 2024

(54) COMPOSITIONS AND METHODS FOR TREATING CEP290 ASSOCIATED DISEASE

(71) Applicant: EDITAS MEDICINE, INC., Cambridge, MA (US)

(72) Inventors: Morgan Lee MAEDER, Jamaican Plain, MA (US); Rina J. MEPANI, Belmont, CA (US); Michael STEFANIDAKIS, Brookline, MA (US)

(73) Assignee: EDITAS MEDICINE, INC., Cambridge, MA (US)

(21) Appl. No.: 18/161,806

(22) Filed: Jan. 30, 2023

Related U.S. Application Data
(63) Continuation of application No. 16/322,922, filed on Aug. 27, 2019, now Pat. No. 11,566,263.
(60) Provisional application No. 62/370,202, filed on Aug. 2, 2016.
(60) Provisional application No. 62/400,526, filed on Sep. 27, 2016.
(60) Provisional application No. 62/443,568, filed on Jan. 6, 2017.
(60) Provisional application No. 62/503,800, filed on May 9, 2017.
(60) Provisional application No. 62/535,193, filed on Jul. 20, 2017.

(51) Int. Cl. C12N 15/90; A61K 9/00; A61K 35/761; C12N 9/22; C12N 15/11; C12N 15/113; C12N 15/86

(52) U.S. Cl. CPC C12N 15/907; A61K 9/0048; A61K 35/761; C12N 9/22; C12N 15/11; C12N 15/113; C12N 15/86; C12N 2310/20; C12N 2320/32; C12N 2330/50; C12N 2330/51; C12N 2750/14143; C12N 2800/80

(57) ABSTRACT

Nucleic acids and viral vectors, particularly adeno-associated virus (AAV) vectors are provided that encode Cas9 and paired guide RNAs. The nucleic acids and vectors, and compositions that comprise them, can be used in methods to treat subjects, to alter cells in subjects who may suffer from an inherited retinal dystrophy such as CEP290 associated disease or who may be in need of alteration of a cell or a cellular nucleic acid sequence associated with an inherited retinal dystrophy such as the CEP290 gene, and/or to treat inherited retinal dystrophies including CEP290 associated disease.

### CROSS REFERENCE TO RELATED APPLICATIONS

The present application is a continuation of U.S. application Ser. No. 16/322,922, filed Aug. 27, 2019, and issuing on Jan. 31, 2023 as U.S. Pat. No. 11,566,263, which is a U.S. national phase application of International Patent Appl. No. PCT/US2017/045191, filed Aug. 2, 2017, which claims the benefit of U.S. Provisional Appl. No. 62/370,202, filed Aug. 2, 2016; U.S. Provisional Appl. No. 62/400,526, filed Sep. 27, 2016; U.S. Provisional Appl. No. 62/443,568, filed Jan. 6, 2017; U.S. Provisional Appl. No. 62/503,800, filed May 9, 2017; and U.S. Provisional Appl. No. 62/535,193, filed Jul. 20, 2017; the contents of which are hereby incorporated by reference in their entirety.

### SEQUENCE LISTING

This application contains a ST.26 compliant Sequence Listing, which was submitted in XML format via Patent Center, and is hereby incorporated by reference in its entirety. The XML copy, created on Mar. 8, 2023, is named Substitute Sequence Listing.xml and is 73,000 B in size.

### FIELD

The disclosure relates to CRISPR/CAS-related methods and components for editing of a target nucleic acid sequence, and applications thereof in connection with CEP290 associated disease.

### BACKGROUND

CEP290 is a 290 kilodalton (kDa) protein encoded by a 90 kilobase-pair (kb) gene, which is thought to be involved in the normal function of the eye and kidney. In cells, the CEP290 protein associates with the centrosome and with cellular scaffold proteins, and is implicated in a variety of cellular processes including cell division, the DNA damage response, and ciliogenesis. Mutations of CEP290 are observed in several diseases, including Senior-Loken syndrome, Meckel Gruber syndrome, Bardet-Biedle syndrome, Joubert Syndrome, and Leber Congenital Amaurosis 10 (LCA10).

LCA10 is an inherited retinal degenerative disease characterized by severe visual impairment or blindness at birth. The disease is inherited in an autosomal recessive fashion and is caused by a C.2991+1655A to G mutation (the “IVS26” mutation) in the CEP290 gene. IVS26 is a loss-of-function mutation in which a cryptic splice donor site is formed in intron 26 of the CEP290 gene, resulting in prematurely truncated CEP290 mRNA transcripts that include an aberrant 128 bp exon. The consequent loss of CEP290 function is thought to disrupt sensory cilia function in photoreceptor cells, leading to the disease.

There are currently no approved therapies for LCA10. Gene therapy strategies for treating CEP290 associated disease are complicated by the size of the protein and the difficulty of packaging large sequences into currently used gene therapy vectors, and no small molecule therapies have been approved for the disease. To address the lack of treatment options for LCA10, the inventors have developed a strategy for correcting the IVS26 mutation in cells using CRISPR/Cas9 genome editing, and have shown deletion of the mutation in up to 60% of HEK293 cells in vitro. See International Patent Publication No. WO2015/138510 by Maeder et al. (“Maeder”), which is incorporated by reference herein.

### SUMMARY

The inventors have addressed a key unmet need in the field by providing new and effective means of delivering genome editing systems to the affected tissues of subjects suffering from CEP290 associated diseases and other inherited retinal dystrophies. This disclosure provides nucleic acids and vectors for efficient transduction of genome editing systems in retinal cells and cells in other tissues, as well as methods of using these vectors to treat subjects. These nucleic acids, vectors and methods represent an important step forward in the development of treatments for CEP290 associated diseases.

In one aspect, the disclosure relates to a method for treating or altering a cell in a subject (e.g., a human subject or an animal subject) that includes administering to the subject a nucleic acid encoding a Cas9 and first and second guide RNAs (gRNAs) targeted to the CEP290 gene of the subject. In certain embodiments, the first and second gRNAs are targeted to one or more target sequences that encompass or are proximal to a CEP290 target position. The first gRNA may include a targeting domain selected from SEQ ID NOS: 1-3 (corresponding RNA sequences in SEQ ID NOS: 26-28, respectively), while the targeting domain of the second gRNA may be selected from SEQ ID NOS: 4-6 (corresponding RNA sequences in SEQ ID NOS: 29-31, respectively). The Cas9, which may be a modified Cas9 (e.g., a Cas9 engineered to alter PAM specificity, improve fidelity, or to alter or improve another structural or functional aspect of the Cas9), may include one or more of a nuclear localization signal (NLS) and/or a polyadenylation signal. Certain embodiments are characterized by Cas9s that include both a C-terminal and an N-terminal NLS. The Cas9 is encoded, in certain embodiments, by SEQ ID NO: 10, and its expression is optionally driven by one of a CMV, EFS, or hGRK1 promoter, as set out in SEQ ID NOS: 13-15 respectively. The nucleic acid also includes, in various cases, first and second inverted terminal repeat sequences (ITRs).

Continuing with this aspect of the disclosure, a nucleic acid comprising any or all of the features described above may be administered to the subject via an adeno-associated viral (AAV) vector, such as an AAV5 vector. The vector may be delivered to the retina of the subject (for example, by subretinal injection). Various embodiments of the method may be used in the treatment of human subjects. For example, the methods may be used to treat subjects suffering from a CEP290 associated disease such as LCA10, to restore CEP290 function in a subject in need thereof, and/or to alter a cell in the subject, such as a retinal cell and/or a photoreceptor cell.

In another aspect, this disclosure relates to a nucleic acid encoding a Cas9, a first gRNA with a targeting domain selected from SEQ ID NOS: 1-3 (corresponding RNA sequences in SEQ ID NOS: 26-28, respectively), and a second gRNA with a targeting domain selected from SEQ ID NOS: 4-6 (corresponding RNA sequences in SEQ ID NOS: 29-31, respectively). The nucleic acid may, in various embodiments, incorporate any or all of the features described above (e.g., the NLS and/or polyadenylation signal; the CMV, EFS or hGRK1 promoter; and/or the ITRs). The nucleic acid may be part of an AAV vector, which vector may be used in medicine, for example to treat a CEP290 associated disease such as LCA10, and/or may be used to edit specific cells including retinal cells, for instance retinal photoreceptor cells. The nucleic acid may also be used for the production of a medicament.

In yet another aspect, this disclosure relates to a method of treating a subject that includes the step of contacting a retina of the subject with one or more recombinant viral vectors (e.g., AAV vectors) that encode a Cas9 and first and second gRNAs. The first and second gRNAs are adapted to form first and second ribonucleoprotein complexes with the Cas9, and the first and second complexes in turn are adapted to cleave first and second target sequences, respectively, on either side of a CEP290 target position as that term is defined below. This cleavage results in the alteration of the nucleic acid sequence of the CEP290 target position. In some embodiments, the step of contacting the retina with one or more recombinant viral vectors includes administering to the retina of the subject, by subretinal injection, a composition comprising the one or more recombinant viral vectors. The alteration of the nucleic acid sequence of the CEP290 target position can include formation of an indel, deletion of part or all of the CEP290 target position, and/or inversion of a nucleotide sequence in the CEP290 target position. The subject, in certain embodiments, is a primate.

The genome editing systems, compositions, and methods of the present disclosure can support high levels of productive editing in retinal cells, e.g., in photoreceptor cells. In certain embodiments, 10%, 15%, 20%, or 25% of retinal cells in samples modified according to the methods of this disclosure (e.g., in retinal samples contacted with a genome editing system of this disclosure) comprise a productive alteration of an allele of the CEP290 gene. A productive alteration may include, variously, a deletion and/or inversion of a sequence comprising an IVS26 mutation, or another modification that results in an increase in the expression of functional CEP290 protein in a cell. In certain embodiments, 25%, 30%, 35%, 40%, 45%, 50%, or more than 50% of photoreceptor cells in retinal samples modified according to the methods of this disclosure (e.g., in retinal samples contacted with a genome editing system of this disclosure) comprise a productive alteration of an allele of the CEP290 gene.

In another aspect, this disclosure relates to a nucleic acid encoding a Cas9 and first and second gRNAs targeted to a CEP290 gene of a subject for use in therapy, e.g. in the treatment of CEP290-associated disease. The CEP290 associated disease may be, in some embodiments, LCA10, and in other embodiments may be selected from the group consisting of Senior-Loken syndrome, Meckel Gruber syndrome, Bardet-Biedle syndrome and Joubert Syndrome. Targeting domains of the first and second gRNAs may comprise the sequences of SEQ ID NOS: 1-3 and NOS: 4-6, respectively, and in certain embodiments the first and second gRNA targeting domains comprise: SEQ ID NOS: 1 and 4. In other embodiments, the first and second gRNA targeting domains comprise the sequences of SEQ ID NOS: 1 and 5, SEQ ID NOS: 1 and 6, SEQ ID NOS: 2 and 4, SEQ ID NOS 3 and 4, or SEQ ID NOS: 3 and 5. In still other embodiments, the first and second targeting domains comprise the sequences of SEQ ID NOS: 2 and 5, SEQ ID NOS: 2 and 6, or SEQ ID NOS: 3 and 6. The gRNAs according to this aspect of the disclosure may be unimolecular, and may comprise RNA sequences according to SEQ ID NO: 7 or SEQ ID NO: 8. Alternatively, the gRNAs may be two-part modular gRNAs according to either sequence, where the crRNA component comprises the portion of SEQ ID NO: 7 or 8 that is underlined, and the tracrRNA component comprises the portion that is double-underlined.

Continuing with this aspect of the disclosure, the Cas9 encoded by the nucleic acid is, in certain embodiments, a Staphylococcus aureus Cas9, which may be encoded by a sequence comprising SEQ ID NO: 10, or having at least 80%, 85%, 90%, 95% or 99% sequence identity thereto. The Cas9 encoded by the nucleic acid may comprise the amino acid sequence of SEQ ID NO: 11 or may share at least 80%, 85%, 90%, 95% or 99% sequence identity therewith. The Cas9 may be modified in some instances, for example to include one or more nuclear localization signals (NLSs) (e.g., a C-terminal and an N-terminal NLS) and/or a polyadenylation signal. Cas9 expression may be driven by a promoter sequence such as the promoter sequence comprising SEQ ID NO: 13, the promoter sequence comprising SEQ ID NO: 14, or the promoter sequence comprising SEQ ID NO: 15.

Staying with this aspect of the disclosure, the promoter sequence for driving the expression of the Cas9 comprises, in certain embodiments, the sequence of a human GRK1 promoter. In other embodiments, the promoter comprises the sequence of a cytomegalovirus (CMV) promoter or an EFS promoter. For example, the nucleic acid may comprise, in various embodiments, a) a CMV promoter for Cas9 and gRNAs comprising (or differing by no more than 3 nucleotides from) targeting domains according to SEQ ID NOs: 1 and 5, or b) a CMV promoter for Cas9 and gRNAs comprising targeting domains according to SEQ ID NOs: 1 and 6, or c) a CMV promoter for Cas9 and gRNAs comprising targeting domains according to SEQ ID NOs: 2 and 4, or d) a CMV promoter for Cas9 and gRNAs comprising targeting domains according to SEQ ID NOs: 3 and 4, or e) a CMV promoter for Cas9 and gRNAs comprising targeting domains according to SEQ ID NOs: 3 and 5, or f) an EFS promoter for Cas9 and gRNAs comprising targeting domains according to SEQ ID NOs: 1 and 5, or g) an EFS promoter for Cas9 and gRNAs comprising targeting domains according to SEQ ID NOs: 1 and 6, or h) an EFS promoter for Cas9 and gRNAs comprising targeting domains according to SEQ ID NOs: 2 and 4, or i) an EFS promoter for Cas9 and gRNAs comprising targeting domains according to SEQ ID NOs: 3 and 4, or j) an EFS promoter for Cas9 and gRNAs comprising targeting domains according to SEQ ID NOs: 3 and 5, or k) an hGRK1 promoter for Cas9 and gRNAs comprising targeting domains according to SEQ ID NOs: 1 and 5, or g) an hGRK1promoter for Cas9 and gRNAs comprising targeting domains according to SEQ ID NOs: 1 and 6, or h) an hGRK1promoter for Cas9 and gRNAs comprising targeting domains according to SEQ ID NOs: 2 and 4, or i) an hGRK1 promoter for Cas9 and gRNAs comprising targeting domains according to SEQ ID NOs: 3 and 4, or j) an hGRK1promoter for Cas9 and gRNAs comprising targeting domains according to SEQ ID NOs: 3 and 5. In other embodiments, the nucleic acid comprises a CMV promoter and guide RNA targeting sequences according to SEQ ID NOS: 1 and 4. In still other embodiments, the nucleic acid comprises an hGRK promoter and guide RNA targeting sequences according to SEQ ID NOS: 2 and 5, or it comprises a CMV promoter and guide RNA targeting sequences according to SEQ ID NOS: 2 and 5, or an hGRK promoter and guide RNA targeting sequences according to SEQ ID NOS: 2 and 6, or it comprises a CMV promoter and guide RNA targeting sequences according to SEQ ID NOS: 3 and 6,. or an hGRK promoter and guide RNA targeting sequences according to SEQ ID NOS: 3 and 6, or it comprises a CMV promoter and guide RNA targeting sequences according to SEQ ID NOS: 2 and 5. And in further embodiments, the promoter is hGRK or CMV while the first and second gRNA targeting domains comprise the sequences of SEQ ID NOS: 1 and 5, SEQ ID NOS: 1 and 6, SEQ ID NOS: 2 and 4, SEQ ID NOS 3 and 4, or SEQ ID NOS: 3 and 5.

In another aspect, the present disclosure relates to adeno-associated virus (AAV) vectors comprising the nucleic acids described above. AAV vectors comprising the foregoing nucleic acids may be administered to a variety of tissues of a subject, though in certain embodiments the AAV vectors are administered to a retina of the subject, and/or are administered by subretinal injection. The AAV vector may comprise an AAV5 capsid.

An additional aspect of this disclosure relates to a nucleic acid as described above, for delivery via an AAV vector also as described above. The nucleic acid includes in some embodiments, first and second inverted terminal repeat sequences (ITRs), a first guide RNA comprising a targeting domain sequence selected from SEQ ID NOS: 1-3, a second guide RNA comprising a targeting domain sequence selected from SEQ ID NOS: 4-6, and a promoter for driving Cas9 expression comprising a sequence selected from SEQ ID NOS: 13-15. In certain embodiments, the nucleic acid includes first and second ITRs and first and second guide RNAs comprising a guide RNA sequence selected from SEQ ID NOS: 7 and 8 (e.g., both first and second guide RNAs comprise the sequence of SEQ ID NO: 8). The nucleic acid may be used in the treatment of human subjects, and/or in the production of a medicament.

The nucleic acids and vectors according to these aspects of the disclosure may be used in medicine, for instance in the treatment of disease. In some embodiments, they are used in the treatment of a CEP290-associated disease, in the treatment of LCA10, or in the treatment of one or more of the following: Senior-Loken syndrome, Meckel Gruber syndrome, Bardet-Biedle syndrome, and/or Joubert Syndrome. Vectors and nucleic acids according to this disclosure may be administered to the retina of a subject, for instance by subretinal injection.

This disclosure also relates to recombinant viral vectors comprising the nucleic acids described above, and to the use of such viral vectors in the treatment of disease. In some embodiments, one or more viral vectors encodes a Cas9, a first gRNA and a second gRNA for use in a method of altering a nucleotide sequence of a CEP 290 target position wherein (a) the first and second gRNAs are adapted to form first and second ribonucleoprotein complexes with the Cas9, and (b) the first and second ribonucleoprotein complexes are adapted to cleave first and second cellular nucleic acid sequences on first and second sides of a CEP290 target position, thereby altering a nucleotide sequence of the CEP290 target position. In use, the one or more recombinant viral vectors is contacted to the retina of a subject, for instance by subretinal injection.

Another aspect of this disclosure relates to AAV vectors, AAV vector genomes and/or nucleic acids that may be carried by AAV vectors, which encode one or more guide RNAs, each comprising a sequence selected from—or having at least 90% sequence identity to—one of SEQ ID NOS: 7 or 8 (corresponding RNA sequences in SEQ ID NOS: 32 and 33, respectively), a sequence encoding a Cas9 and a promoter sequence operably coupled to the Cas9 coding sequence, which promoter sequence comprises a sequence selected from—or having at least 90% sequence identity to—one of SEQ ID NOS: 13-15. The Cas9 coding sequence may comprise the sequence of SEQ ID NO: 10, or it may share at least 90% sequence identity therewith. Alternatively or additionally, the Cas9 coding sequence may encode an amino acid sequence comprising SEQ ID NO: 11, or sharing at least 90% sequence identity therewith. In certain embodiments, the AAV vector, vector genome or nucleic acid further comprises one or more of the following: left and right ITR sequences, optionally selected from—or having at least 90% sequence identity to— SEQ ID NOS: 16 and 17, respectively; and one or more U6 promoter sequences operably coupled to the one or more guide RNA sequences. The U6 promoter sequences may comprise, or share at least 90% sequence identity with, SEQ ID NO: 9.

This listing is intended to be exemplary and illustrative rather than comprehensive and limiting. Additional aspects and embodiments may be set out in, or apparent from, the remainder of this disclosure and the claims.

### DESCRIPTION OF THE DRAWINGS

The accompanying drawings exemplify certain aspects and embodiments of the present disclosure. The depictions in the drawings are intended to provide illustrative, and schematic rather than comprehensive, examples of certain aspects and embodiments of the present disclosure. The drawings are not intended to be limiting or binding to any particular theory or model, and are not necessarily to scale. Without limiting the foregoing, nucleic acids and polypeptides may be depicted as linear sequences, or as schematic, two- or three-dimensional structures; these depictions are intended to be illustrative, rather than limiting or binding to any particular model or theory regarding their structure.

FIGS. 1A-C include schematic depictions of exemplary AAV viral genome according to certain embodiments of the disclosure. FIG. 1A shows an AAV genome for use in altering a CEP290 target position which encodes, inter alia, two guide RNAs having specific targeting domains selected from SEQ ID NOS: 1-3 and 4-6 and an S. aureus Cas9.

FIG. 1B shows an AAV genome that may be used for a variety of applications, including without limitation the alteration of the CEP290 target position, encoding two guide RNAs comprising the sequences of SEQ ID NOS. 7 and/or 8 and an S. aureus Cas9. FIG. 1C shows an AAV genome encoding one or two guide RNAs, each driven by a U6 promoter, and an S. aureus Cas9. In the figure, N may be 1 or two.

FIG. 2 illustrates the genome editing strategy implemented in certain embodiments of this disclosure.

FIG. 3 schematically depicts a gRNA used in certain embodiments of the disclosure.

FIG. 4A shows a photomicrograph of a mouse retinal explant on a support matrix; retinal tissue is indicated by the arrow. FIG. 4B shows a fluorescence micrograph from a histological section of a mouse retinal explant illustrating AAV transduction of cells in multiple retinal layers with a GFP reporter. FIG. 4C shows a micrograph from a histological section of a primate retinal tissue treated with vehicle. FIG. 4D shows a micrograph from a histological section of a primate retinal tissue treated with AAV5 vector encoding S. aureus Cas9 operably linked to the photoreceptor-specific hGRK1 promoter. Dark staining in the outer nuclear layer (ONL) indicates that cells were successfully transduced with AAV and express Cas9.

FIG. 5A and FIG. 5B show expression of Cas9 mRNA and gRNA, respectively, normalized to GAPDH mRNA expression. UT denotes untreated; GRK1-Cas refers to a vector in which Cas9 expression is driven by the photoreceptor-specific hGRK1 promoter; dCMV-Cas and EFS-Cas similarly refer to vectors in which Cas9 expression is driven by the dCMV promoter or the EFS promoter. Conditions in which gRNAs are included in the vector are denoted by the bar captioned “with gRNA.” Light and dark bars depict separate experimental replicates.

FIG. 6 summarizes the edits observed in mouse retinal explants 7 days after transduction with AAV5-mCEPgRNAs-Cas9. Edits were binned into one of three categories: no edit, indel at one of two guide sites, and deletion of sequence between the guide sites. Each bar graph depicts the observed edits as a percentage of sequence reads from individual explants transduced with AAV vectors in which Cas9 was driven by the promoter listed (hGRK1, CMV or EFS).

FIG. 7 summarizes the edits observed in the CEP290 gene in retinal punch samples obtained from cynomolgus monkeys treated with AAV vectors encoding genome editing systems according to the present disclosure.

FIG. 8A depicts a reporter construct that was used to assess the effect of certain editing outcomes, including inversions and deletions, on the IVS26 splicing defect. FIG. 8B depicts the relative levels of GFP reporter expression in WT, IVS26, deletion and inversion conditions, normalized to mCherry expression.

FIG. 9 summarizes the productive CEP290 edits observed in human retinal explants 14 or 28 days after transduction with AAV vectors in which Cas9 was driven by the promoter listed (hGRK1 or CMV).

### DETAILED DESCRIPTION

### Definitions and Abbreviations

Unless otherwise specified, each of the following terms has the meaning set forth in this section.

The indefinite articles “a” and “an” denote at least one of the associated noun, and are used interchangeably with the terms “at least one” and “one or more.” For example, the phrase “a module” means at least one module, or one or more modules.

The conjunctions “or” and “and/or” are used interchangeably.

“Domain” is used to describe a segment of a protein or nucleic acid. Unless otherwise indicated, a domain is not required to have any specific functional property.

An “indel” is an insertion and/or deletion in a nucleic acid sequence. An indel may be the product of the repair of a DNA double strand break, such as a double strand break formed by a genome editing system of the present disclosure. An indel is most commonly formed when a break is repaired by an “error prone” repair pathway such as the NHEJ pathway described below. Indels are typically assessed by sequencing (most commonly by “next-gen” or “sequencing-by-synthesis” methods, though Sanger sequencing may still be used) and are quantified by the relative frequency of numerical changes (e.g., ±1, ±2 or more bases) at a site of interest among all sequencing reads. DNA samples for sequencing can be prepared by a variety of methods known in the art, and may involve the amplification of sites of interest by polymerase chain reaction (PCR) or the capture of DNA ends generated by double strand breaks, as in the GUIDEseq process described in Tsai 2016 (incorporated by reference herein). Other sample preparation methods are known in the art. Indels may also be assessed by other methods, including in situ hybridization methods such as the FiberComb™ system commercialized by Genomic Vision (Bagneux, France), and other methods known in the art.

“CEP290 target position” and “CEP290 target site” are used interchangeably herein to refer to a nucleotide or nucleotides in or near the CEP290 gene that are targeted for alteration using the methods described herein. In certain embodiments, a mutation at one or more of these nucleotides is associated with a CEP290 associated disease. The terms “CEP290 target position” and “CEP290 target site” are also used herein to refer to these mutations. For example, the IVS26 mutation is one non-limiting embodiment of a CEP290 target position/target site.

“Non-homologous end joining” or “NHEJ” as used herein refers to ligation mediated repair and/or non-template mediated repair including canonical NHEJ (cNHEJ), alternative NHEJ (altNHEJ), microhomology-mediated end joining (MMEJ) and synthesis-dependent microhomology-mediated end joining (SD-MMEJ).

“Replacement” or “replaced” as used herein with reference to a modification of a molecule does not require a process limitation but merely indicates that the replacement entity is present.

“Subject” means a human, mouse, or non-human primate. A human subject can be any age (e.g., an infant, child, young adult, or adult), and may suffer from a disease, or may be in need of alteration of a gene.

“Treat,” “treating,” and “treatment” as used herein mean the treatment of a disease in a subject (e.g., a human subject), including one or more of inhibiting the disease, i.e., arresting or preventing its development or progression; relieving the disease, i.e., causing regression of the disease state; relieving one or more symptoms of the disease; and curing the disease.

“Prevent,” “preventing,” and “prevention” as used herein means the prevention of a disease in a subject, e.g., in a human, including (a) avoiding or precluding the disease; (b) affecting the predisposition toward the disease; (c) preventing or delaying the onset of at least one symptom of the disease.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. A nucleotide sequence typically carries genetic information, including the information used by cellular machinery to make proteins and enzymes. These terms include double- or single-stranded genomic DNA, RNA, any synthetic and genetically manipulated polynucleotide, and both sense and antisense polynucleotides. This also includes nucleic acids containing modified bases.

Conventional IUPAC notation is used in nucleotide sequences presented herein, as shown in Table 1, below (see also Cornish-Bowden 1985, incorporated by reference herein). It should be noted, however, that “T” denotes “Thymine or Uracil” insofar as a given sequence (such as a gRNA sequence) may be encoded by either DNA or RNA.

TABLE 1
IUPAC nucleic acid notation

| Character | Base |
|---|---|
| A | Adenine |
| T | Thymine |
| G | Guanine |
| C | Cytosine |
| U | Uracil |
| K | G or T/U |
| M | A or C |
| R | A or G |
| Y | C or T/U |
| S | C or G |
| W | A or T/U |
| B | C, G, or T/U |
| V | A, C, or G |
| H | A, C, or T/U |
| D | A, G, or T/U |
| N | A, C, G, or T/U |

The terms “protein,” “peptide” and “polypeptide” are used interchangeably to refer to a sequential chain of amino acids linked together via peptide bonds. The terms include individual proteins, groups or complexes of proteins that associate together, as well as fragments, variants, derivatives and analogs of such proteins. Peptide sequences are presented using conventional notation, beginning with the amino or N-terminus on the left, and proceeding to the carboxyl or C-terminus on the right. Standard one-letter or three-letter abbreviations may be used.

### Overview

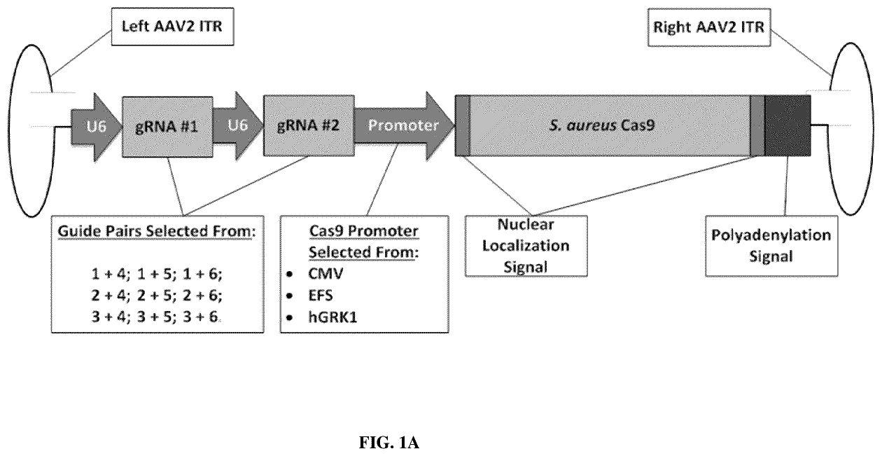

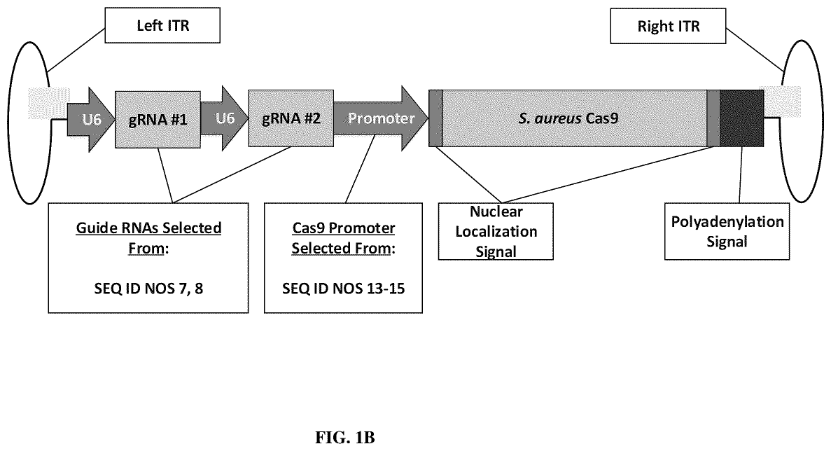

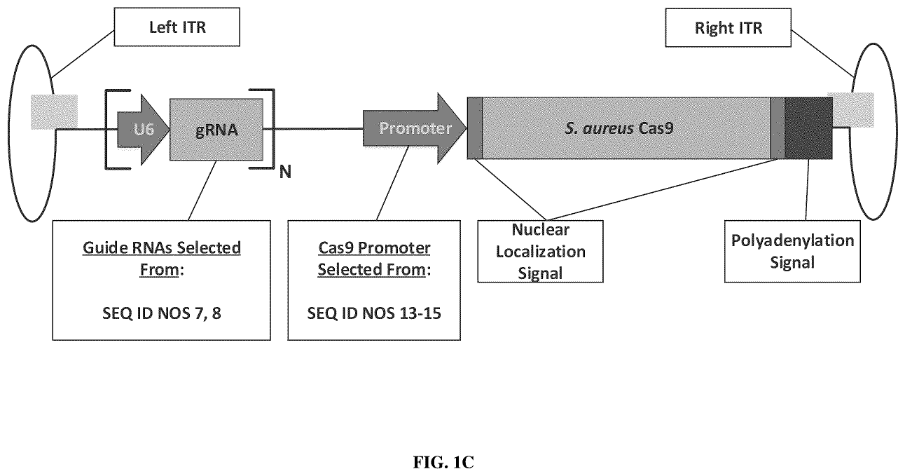

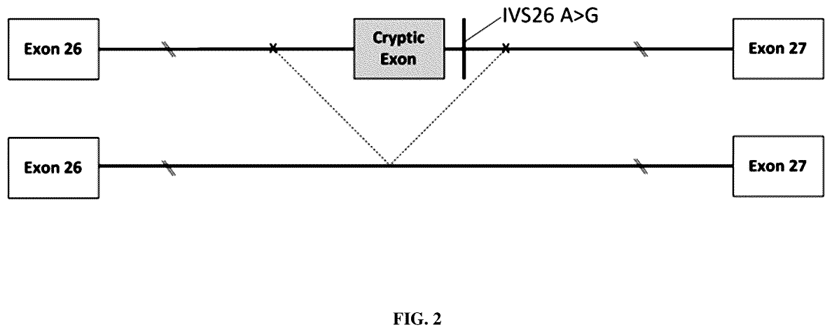

In certain aspects, the present disclosure focuses on AAV vectors encoding CRISPR/Cas9 genome editing systems, and on the use of such vectors to treat CEP290 associated disease. Exemplary AAV vector genomes are schematized in FIGS. 1A through 1C, which illustrates certain fixed and variable elements of these vectors: inverted terminal repeats (ITRs), one or two gRNA sequences and promoter sequences to drive their expression, a Cas9 coding sequence and another promoter to drive its expression. Each of these elements is discussed in detail below.

Turning first to the gRNA pairs utilized in the nucleic acids or AAV vectors of the present disclosure, one of three “left” or “upstream” guides may be used to cut upstream (between exon 26 and the IVS26 mutation), and one of three “right” or “downstream” guides is used to cut downstream (between the IVS26 mutation and exon 27). Targeting domain sequences of these guides are presented in Table 2, below:

TABLE 2
Upstream (left) and Downstream (right) gRNA Targeting Domain Sequences
Upstream (left) guides

| Targeting | | |
|---|---|---|
| domain | DNA | RNA |
| CEP290- | GTTCTGTCCTCAGTAAAAGGTA | GUUCUGUCCUCAGUAAAAGGUA |
| 323 | (SEQ ID NO: 1) | (SEQ ID NO: 26) |
| CEP290- | GAATAGTTTGTTCTGGGTAC | GAAUAGUUUGUUCUGGGUAC |
| 490 | (SEQ ID NO: 2) | (SEQ ID NO: 27) |
| CEP290- | GAGAAAGGGATGGGCACTTA | GAGAAAGGGAUGGGCACUUA |
| 492 | (SEQ ID NO: 3) | (SEQ ID NO: 28) |

Downstream (right) guides

| Targeting | | |
|---|---|---|
| domain | DNA | RNA |
| CEP290- | GTCAAAAGCTACCGGTTACCTG | GUCAAAAGCUACCGGUUACCUG |
| 64 | (SEQ ID NO: 4) | (SEQ ID NO: 29) |
| CEP290- | GATGCAGAACTAGTGTAGAC | GAUGCAGAACUAGUGUAGAC |
| 496 | (SEQ ID NO: 5) | (SEQ ID NO: 30) |
| CEP290- | GAGTATCTCCTGTTTGGCA | GAGUAUCUCCUGUUUGGCA |
| 504 | (SEQ ID NO: 6) | (SEQ ID NO: 31) |

The left and right guides can be used in any combination, though certain combinations may be more suitable for certain applications. Table 3 sets forth several upstream+downstream guide pairs used in the embodiments of this disclosure. It should be noted, notwithstanding the use of “left” and “right” as nomenclature for gRNAs, that any guide in a pair, upstream or downstream, may be placed in either one of the gRNA coding sequence positions illustrated in FIG. 1.

TABLE 3
Upstream (Left) + Downstream (Right) Guide Pairs

| | | | Downstream | | |
|---|---|---|---|---|---|
| | | | 4 | 5 | 6 |
| U | Upstream | 1 | 1 + 4 | 1 + 5 | 1 + 6 |
| | | 2 | 2 + 4 | 2 + 5 | 2 + 6 |
| | | 3 | 3 + 4 | 3 + 5 | 3 + 6 |

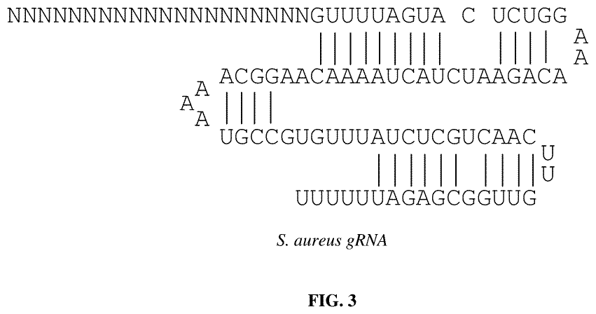

In some embodiments, the gRNAs used in the present disclosure are derived from S. aureus gRNAs and can be unimolecular or modular, as described below. An exemplary unimolecular S. aureus gRNA is shown in FIG. 3, and exemplary DNA and RNA sequences corresponding to unimolecular S. aureus gRNAs are shown below:

DNA:
(SEQ ID NO: 7)
[N]₁₆₋₂₄GTTTTAGTACTCTGGAAACAGAATCTACTAAAACAAGGCAAA
ATGCCGTGTTTATCTCGTCAACTTGTTGGCGAGATTTTTT
and
RNA:
(SEQ ID NO: 32)
[N]₁₆₋₂₄GUUUUAGUACUCUGGAAACAGAAUCUACUAAAACAAGGCAAA
AUGCCGUGUUUAUCUCGUCAACUUGUUGGCGAGAUUUUUU.
DNA:
(SEQ ID NO: 8)
[N]₁₆₋₂₄GTTATAGTACTCTGGAAACAGAATCTACTATAACAAGGCAAA
ATGCCGTGTTTATCTCGTCAACTTGTTGGCGAGATTTTTT
and
RNA:
(SEQ ID NO: 33)
[N]₁₆₋₂₄GUUAUAGUACUCUGGAAACAGAAUCUACUAUAACAAGGCAAA
AUGCCGUGUUUAUCUCGUCAACUUGUUGGCGAGAUUUUUU.

It should be noted that, while the figure depicts a targeting domain of 20 nucleotides, the targeting domain can have any suitable length. gRNAs used in the various embodiments of this disclosure preferably include targeting domains of between 16 and 24 (inclusive) bases in length at their 5′ ends, and optionally include a 3′ U6 termination sequence as illustrated.

The gRNA in FIG. 3 is depicted as unimolecular, but in some instances modular guides can be used. In the exemplary unimolecular gRNA sequences above, a 5′ portion corresponding to a crRNA (underlined) is connected by a GAAA linker to a 3′ portion corresponding to a tracrRNA (double underlined). Skilled artisans will appreciate that two-part modular gRNAs can be used that correspond to the underlined and double underlined sections.

Either one of the gRNAs presented above can be used with any of targeting sequences 1-6, and two gRNAs in a pair do not necessarily include the same backbone sequence. Additionally, skilled artisans will appreciate that the exemplary gRNA designs set forth herein can be modified in a variety of ways, which are described below or are known in the art; the incorporation of such modifications is within the scope of this disclosure.

Expression of each of the gRNAs in the AAV vector is driven by a pair of U6 promoters, such as a human U6 promoter. An exemplary U6 promoter sequence, as set forth in Maeder, is presented below:

(SEQ ID NO: 9)
AAGGTCGGGCAGGAAGAGGGCCTATTTCCCATGATTCCTTCATATTTGCA
TATACGATACAAGGCTGTTAGAGAGATAATTAGAATTAATTTGACTGTAA
ACACAAAGATATTAGTACAAAATACGTGACGTAGAAAGTAATAATTTCTT
GGGTAGTTTGCAGTTTTAAAATTATGTTTTAAAATGGACTATCATATGCT
TACCGTAACTTGAAAGTATTTCGATTTCTTGGCTTTATATATCTTGTGGA
AAGGACGAAACACC.

Turning next to Cas9, in some embodiments the Cas9 protein is S. aureus Cas9. In further embodiments of this disclosure an S. aureus Cas9 sequence is modified to include two nuclear localization sequences (NLSs) at the C- and N-termini of the Cas9 protein, and a mini-polyadenylation signal (or Poly-A sequence). Exemplary S. aureus Cas9 sequences (both nucleotide and peptide) are shown below:

TABLE 4
saCas9 Sequences

| | |
|---|---|
| Codon- | ATGAAAAGGAACTACATTCTGGGGCTGGACATCGGGATTACAAGCGTGGGG |
| optimized | TATGGGATTATTGACTATGAAACAAGGGACGTGATCGACGCAGGCGTCAGA |
| S. aureus | CTGTTCAAGGAGGCCAACGTGGAAAACAATGAGGGACGGAGAAGCAAGAG |
| Cas9 | GGGAGCCAGGCGCCTGAAACGACGGAGAAGGCACAGAATCCAGAGGGTGA |
| nucleotide | AGAAACTGCTGTTCGATTACAACCTGCTGACCGACCATTCTGAGCTGAGTGG |
| (SEQ ID | AATTAATCCTTATGAAGCCAGGGTGAAAGGCCTGAGTCAGAAGCTGTCAGA |
| NO: 10) | GGAAGAGTTTTCCGCAGCTCTGCTGCACCTGGCTAAGCGCCGAGGAGTGCAT |
| | AACGTCAATGAGGTGGAAGAGGACACCGGCAACGAGCTGTCTACAAAGGAA |
| | CAGATCTCACGCAATAGCAAAGCTCTGGAAGAGAAGTATGTCGCAGAGCTG |
| | CAGCTGGAACGGCTGAAGAAAGATGGCGAGGTGAGAGGGTCAATTAATAGG |
| | TTCAAGACAAGCGACTACGTCAAAGAAGCCAAGCAGCTGCTGAAAGTGCAG |
| | AAGGCTTACCACCAGCTGGATCAGAGCTTCATCGATACTTATATCGACCTGC |
| | TGGAGACTCGGAGAACCTACTATGAGGGACCAGGAGAAGGGAGCCCCTTCG |
| | GATGGAAAGACATCAAGGAATGGTACGAGATGCTGATGGGACATTGCACCT |
| | ATTTTCCAGAAGAGCTGAGAAGCGTCAAGTACGCTTATAACGCAGATCTGTA |
| | CAACGCCCTGAATGACCTGAACAACCTGGTCATCACCAGGGATGAAAACGA |
| | GAAACTGGAATACTATGAGAAGTTCCAGATCATCGAAAACGTGTTTAAGCAG |
| | AAGAAAAAGCCTACACTGAAACAGATTGCTAAGGAGATCCTGGTCAACGAA |
| | GAGGACATCAAGGGCTACCGGGTGACAAGCACTGGAAAACCAGAGTTCACC |
| | AATCTGAAAGTGTATCACGATATTAAGGACATCACAGCACGGAAAGAAATC |
| | ATTGAGAACGCCGAACTGCTGGATCAGATTGCTAAGATCCTGACTATCTACC |
| | AGAGCTCCGAGGACATCCAGGAAGAGCTGACTAACCTGAACAGCGAGCTGA |
| | CCCAGGAAGAGATCGAACAGATTAGTAATCTGAAGGGGTACACCGGAACAC |
| | ACAACCTGTCCCTGAAAGCTATCAATCTGATTCTGGATGAGCTGTGGCATAC |
| | AAACGACAATCAGATTGCAATCTTTAACCGGCTGAAGCTGGTCCCAAAAAAG |
| | GTGGACCTGAGTCAGCAGAAAGAGATCCCAACCACACTGGTGGACGATTTC |
| | ATTCTGTCACCCGTGGTCAAGCGGAGCTTCATCCAGAGCATCAAAGTGATCA |
| | ACGCCATCATCAAGAAGTACGGCCTGCCCAATGATATCATTATCGAGCTGGC |
| | TAGGGAGAAGAACAGCAAGGACGCACAGAAGATGATCAATGAGATGCAGA |
| | AACGAAACCGGCAGACCAATGAACGCATTGAAGAGATTATCCGAACTACCG |
| | GGAAAGAGAACGCAAAGTACCTGATTGAAAAAATCAAGCTGCACGATATGC |
| | AGGAGGGAAAGTGTCTGTATTCTCTGGAGGCCATCCCCCTGGAGGACCTGCT |
| | GAACAATCCATTCAACTACGAGGTCGATCATATTATCCCCAGAAGCGTGTCC |
| | TTCGACAATTCCTTTAACAACAAGGTGCTGGTCAAGCAGGAAGAGAACTCTA |
| | AAAAGGGCAATAGGACTCCTTTCCAGTACCTGTCTAGTTCAGATTCCAAGAT |
| | CTCTTACGAAACCTTTAAAAAGCACATTCTGAATCTGGCCAAAGGAAAGGGC |
| | CGCATCAGCAAGACCAAAAAGGAGTACCTGCTGGAAGAGCGGGACATCAAC |
| | AGATTCTCCGTCCAGAAGGATTTTATTAACCGGAATCTGGTGGACACAAGAT |
| | ACGCTACTCGCGGCCTGATGAATCTGCTGCGATCCTATTTCCGGGTGAACAA |
| | TCTGGATGTGAAAGTCAAGTCCATCAACGGCGGGTTCACATCTTTTCTGAGG |
| | CGCAAATGGAAGTTTAAAAAGGAGCGCAACAAAGGGTACAAGCACCATGCC |
| | GAAGATGCTCTGATTATCGCAAATGCCGACTTCATCTTTAAGGAGTGGAAAA |
| | AGCTGGACAAAGCCAAGAAAGTGATGGAGAACCAGATGTTCGAAGAGAAGC |
| | AGGCCGAATCTATGCCCGAAATCGAGACAGAACAGGAGTACAAGGAGATTT |
| | TCATCACTCCTCACCAGATCAAGCATATCAAGGATTTCAAGGACTACAAGTA |
| | CTCTCACCGGGTGGATAAAAAGCCCAACAGAGACiCTGATCAATGACACCCT |
| | GTATAGTACAAGAAAAGACGATAAGGGGAATACCCTGATTGTGAACAATCT |
| | GAACGGACTGTACGACAAAGATAATGACAAGCTGAAAAAGCTGATCAACAA |
| | AAGTCCCGAGAAGCTGCTGATGTACCACCATGATCCTCAGACATATCAGAAA |
| | CTGAAGCTGATTATGGAGCAGTACGGCGACGAGAAGAACCCACTGTATAAG |
| | TACTATGAAGAGACTGGGAACTACCTGACCAAGTATAGCAAAAAGGATAAT |
| | GGCCCCGTGATCAAGAAGATCAAGTACTATGGGAACAAGCTGAATGCCCAT |
| | CTGGACATCACAGACGATTACCCTAACAGTCGCAACAAGGTGGTCAAGCTGT |
| | CACTGAAGCCATACAGATTCGATGTCTATCTGGACAACGGCGTGTATAAATT |
| | TGTGACTGTCAAGAATCTGGATGTCATCAAAAAGGAGAACTACTATGAAGTG |
| | AATAGCAAGTGCTACGAAGAGGCTAAAAAGCTGAAAAAGATTAGCAACCAG |
| | GCAGAGTTCATCGCCTCCTTTTACAACAACGACCTGATTAAGATCAATGGCG |
| | AACTGTATAGGGTCATCGGGGTGAACAATGATCTGCTGAACCGCATTGAAGT |
| | GAATATGATTGACATCACTTACCGAGAGTATCTGGAAAACATGAATGATAAG |
| | CGCCCCCCTCGAATTATCAAAACAATTGCCTCTAAGACTCAGAGTATCAAAA |
| | AGTACTCAACCGACATTCTGGGAAACCTGTATGAGGTGAAGAGCAAAAAGC |
| | ACCCTCAGATTATCAAAAAGGGC |
| S. aureus | MKRNYILGLDIGITSVGYGIIDYETRDVIDAGVRLFKEANVENNEGRRSKRGARR |
| Cas9 | LKRRRRHRIQRVKKLLFDYNLLTDHSELSGINPYEARVKGLSQKLSEEEFSAALL |
| protein | HLAKRRGVHNVNEVEEDTGNELSTKEQISRNSKALEEKYVAELQLERLKKDGE |
| (SEQ ID | VRGSINRFKTSDYVKEAKQLLKVQKAYHQLDQSFIDTYIDLLETRRTYYEGPGE |
| NO: 11) | GSPFGWKDIKEWYEMLMGHCTYFPEELRSVKYAYNADLYNALNDLNNLVITRD |
| | ENEKLEYYEKFQIIENVFKQKKKPTLKQIAKEILVNEEDIKGYRVTSTGKPEFTNL |
| | KVYHDIKDITARKEIIENAELLDQIAKILTIYQSSEDIQEELTNLNSELTQEEIEQISN |
| | LKGYTGTHNLSLKAINLILDELWHTNDNQIAIFNRLKLVPKKVDLSQQKEIPTTL |
| | VDDFILSPVVKRSFIQSIKVINAIIKKYGLPNDIIIELAREKNSKDAQKMINEMQKR |
| | NRQTNERIEEIIRTTGKENAKYLIEKIKLHDMQEGKCLYSLEAIPLEDLLNNPFNY |
| | EVDHIIPRSVSFDNSFNNKVLVKQEENSKKGNRTPFQYLSSSDSKISYETFKKHIL |
| | NLAKGKGRISKTKKEYLLEERDINRFSVQKDFINRNLVDTRYATRGLMNLLRSY |
| | FRVNNLDVKVKSINGGFTSFLRRKWKFKKERNKGYKHHAEDALIIANADFIFKE |
| | WKKLDKAKKVMENQMFEEKQAESMPEIETEQEYKEIFITPHQIKHIKDFKDYKY |
| | SHRVDKKPNRELINDTLYSTRKDDKGNTLIVNNLNGLYDKDNDKLKKLINKSPE |
| | KLLMYHHDPQTYQKLKLIMEQYGDEKNPLYKYYEETGNYLTKYSKKDNGPVIK |
| | KIKYYGNKLNAHLDITDDYPNSRNKVVKLSLKPYRFDVYLDNGVYKFVTVKNL |
| | DVIKKENYYEVNSKCYEEAKKLKKISNQAEFIASFYNNDLIKINGELYRVIGVNN |
| | DLLNRIEVNMIDITYREYLENMNDKRPPRIIKTIASKTQSIKKYSTDILGNLYEVKS |
| | KKHPQIIKKG |

These sequences are exemplary in nature, and are not intended to be limiting. The skilled artisan will appreciate that modifications of these sequences may be possible or desirable in certain applications; such modifications are described below, or are known in the art, and are within the scope of this disclosure.

Skilled artisans will also appreciate that polyadenylation signals are widely used and known in the art, and that any suitable polyadenylation signal can be used in the embodiments of this disclosure. One exemplary polyadenylation signal is set forth below:

(SEQ ID NO: 12)
TAGCAATAAAGGATCGTTTATTTTCATTGGAAGCGTGTGTTGGTTTTTTG
ATCAGGCGCG.

Cas9 expression is driven, in certain vectors of this disclosure, by one of three promoters: cytomegalovirus (CMV), elongation factor-1 (EFS), or human g-protein receptor coupled kinase-1 (hGRK1), which is specifically expressed in retinal photoreceptor cells. Nucleotide sequences for each of these promoters are provided in Table 5. Modifications of these sequences may be possible or desirable in certain applications, and such modifications are within the scope of this disclosure.

TABLE 5
Cas9 Promoter Sequences

| | |
|---|---|
| CMV | CATTGATTATTGACTAGTTATTAATAGTAATCAATTACGGGGTCATTAG |
| (SEQ ID NO: 13) | TTCATAGCCCATATATGGAGTTCCGCGTTACATAACTTACGGTAAATGG |
| | CCCGCCTGGCTGACCGCCCAACGACCCCCGCCCATTGACGTCAATAAT |
| | GACGTATGTTCCCATAGTAACGCCAATAGGGACTTTCCATTGACGTCAA |
| | TGGGTGGACTATTTACGGTAAACTGCCCACTTGGCAGTACATCAAGTGT |
| | ATCATATGCCAAGTACGCCCCCTATTGACGTCAATGACGGTAAATGGC |
| | CCGCCTGGCATTATGCCCAGTACATGACCTTATGGGACTTTCCTACTTG |
| | GCAGTACATCTACGTATTAGTCATCGCTATTACCATGGTGATGCGGTTT |
| | TGGCAGTACATCAATGGGCGTGGATAGCGGTTTGACTCACGGGGATTT |
| | CCAAGTCTCCACCCCATTGACGTCAATGGGAGTTTGTTTTGGCACCAAA |
| | ATCAACGGGACTTTCCAAAATGTCGTAACAACTCCGCCCCATTGACGC |
| | AAATGGGCGGTAGGCGTGTACGGTGGGAGGTCTATATAAGCAGAGCTG |
| | GTTTAGTGAACCGTCAGATCCGCTAGAGATCCGC |
| EFS | TCGAGTGGCTCCGGTGCCCGTCAGTGGGCAGAGCGCACATCGCCCACA |
| (SEQ ID NO: 14) | GTCCCCGAGAAGTTGGGGGGAGGGGTCGGCAATTGAACCGGTGCCTAG |
| | AGAAGGTGGCGCGGGGTAAACTGGGAAAGTGATGTCGTGTACTGGCTC |
| | CGCCTTTTTCCCGAGGGTGGGGGAGAACCGTATATAAGTGCAGTAGTC |
| | GCCGTGAACGTTCTTTTTCGCAACGGGTTTGCCGCCAGAACACAGGTGT |
| | CGTGACCGCGG |
| hGRK1 | GGGCCCCAGAAGCCTGGTGGTTGTTTGTCCTTCTCAGGGGAAAAGTGA |
| (SEQ ID NO: 15) | GGCGGCCCCTTGGAGGAAGGGGCCGGGCAGAATGATCTAATCGGATTC |
| | CAAGCAGCTCAGGGGATTGTCTTTTTCTAGCACCTTCTTGCCACTCCTA |
| | AGCGTCCTCCGTGACCCCGGCTGGGATTTCGCCTGGTGCTGTGTCAGCC |
| | CCGGTCTCCCAGGGGCTTCCCAGTGGTCCCCAGGAACCCTCGACAGGG |
| | CCCGGTCTCTCTCGTCCAGCAAGGGCAGGGACGGGCCACAGGCCAAGG |
| | GC |

AAV genomes according to the present disclosure generally incorporate inverted terminal repeats (ITRs) derived from the AAV2 serotype. Exemplary left and right ITRs are presented in Table 6. It should be noted, however, that numerous modified versions of the AAV2 ITRs are used in the field, and the ITR sequences shown below are exemplary and are not intended to be limiting. Modifications of these sequences are known in the art, or will be evident to skilled artisans, and are thus included in the scope of this disclosure.

TABLE 6
AAV2 ITR Sequences

| | |
|---|---|
| AAV2 | TTGGCCACTCCCTCTCTGCGCGCTCGCTCGCTCACTGAGG |
| Left | CCGGGCGACCAAAGGTCGCCCGACGCCCGGGCTTTGCCC |
| ITR | GGGCGGCCTCAGTGAGCGAGCGAGCGCGCAGAGAGGGA |
| (SEQ ID | GTGGCCAACTCCATCACTAGGGGTTCCT |
| NO: 16) | |
| AAV2 | AGGAACCCCTAGTGATGGAGTTGGCCACTCCCTCTCTGCG |
| Right | CGCTCGCTCGCTCACTGAGGCCGCCCGGGCAAAGCCCGG |
| ITR | GCGTCGGGCGACCTTTGGTCGCCCGGCCTCAGTGAGCGA |
| (SEQ ID | GCGAGCGCGCAGAGAGGGAGTGGCCAA |
| NO: 17) | |

As FIG. 1 illustrates, the gRNA pairs and the Cas9 promoter are variable and can be selected from the lists presented above. For clarity, this disclosure encompasses nucleic acids and/or AAV vectors comprising any combination of these elements, though certain combinations may be preferred for certain applications. Accordingly, in various embodiments of this disclosure, a nucleic acid or AAV vector encodes a CMV promoter for the Cas9, and gRNAs comprising targeting domains according to SEQ ID NOS: 1 and 4; a CMV promoter and gRNAs comprising targeting domains according to SEQ ID NOS: 1 and 5; a CMV promoter and gRNAs comprising targeting domains according to SEQ ID NOS: 1 and 6; a CMV promoter and gRNAs comprising targeting domains according to SEQ ID NOS: 2 and 4; a CMV promoter and gRNAs comprising targeting domains according to SEQ ID NOS: 2 and 5; a CMV promoter and gRNAs comprising targeting domains according to SEQ ID NOS: 2 and 6; a CMV promoter and gRNAs comprising targeting domains according to SEQ ID NOS: 3 and 4; a CMV promoter and gRNAs comprising targeting domains according to SEQ ID NOS: 3 and 5; a CMV promoter and gRNAs comprising targeting domains according to SEQ ID NOS: 3 and 6; an EFS promoter and gRNAs comprising targeting domains according to SEQ ID NOS: 1 and 4; an EFS promoter and gRNAs comprising targeting domains according to SEQ ID NOS: 1 and 5; an EFS promoter and gRNAs comprising targeting domains according to SEQ ID NOS: 1 and 6; an EFS promoter and gRNAs comprising targeting domains according to SEQ ID NOS: 2 and 4; an EFS promoter and gRNAs comprising targeting domains according to SEQ ID NOS: 2 and 5; an EFS promoter and gRNAs comprising targeting domains according to SEQ ID NOS: 2 and 6; an EFS promoter and gRNAs comprising targeting domains according to SEQ ID NOS: 3 and 4; an EFS promoter and gRNAs comprising targeting domains according to SEQ ID NOS: 3 and 5; an EFS promoter and gRNAs comprising targeting domains according to SEQ ID NOS: 3 and 6; an hGRK1 promoter and gRNAs comprising targeting domains according to SEQ ID NOS: 1 and 4; an hGRK1 promoter and gRNAs comprising targeting domains according to SEQ ID NOS: 1 and 5; an hGRK1 promoter and gRNAs comprising targeting domains according to SEQ ID NOS: 1 and 6; an hGRK1 promoter and gRNAs comprising targeting domains according to SEQ ID NOS: 2 and 4; an hGRK1 promoter and gRNAs comprising targeting domains according to SEQ ID NOS: 2 and 5; an hGRK1 promoter and gRNAs comprising targeting domains according to SEQ ID NOS: 2 and 6; an hGRK1 promoter and gRNAs comprising targeting domains according to SEQ ID NOS: 3 and 4; an hGRK1 promoter and gRNAs comprising targeting domains according to SEQ ID NOS: 3 and 5; or an hGRK1 promoter and gRNAs comprising targeting domains according to SEQ ID NOS: 3 and 6.

In various embodiments, the nucleic acid or AAV vector encodes the following: left and right AAV2 ITR sequences, a first U6 promoter to drive expression of a first guide RNA having a sequence selected from SEQ ID NOS: 7 and 8 (corresponding RNA sequences in SEQ ID NOs: 32, and 33, respectively) and/or comprising a targeting domain sequence according to one of SEQ ID NOS: 1-3 (corresponding RNA sequences in SEQ ID NOs: 26-28, respectively), a second U6 promoter to drive expression of a second guide RNA comprising a sequence selected from SEQ ID NOS: 7 and 8 and/or comprising a targeting domain sequence according to one of SEQ ID NOS: 4-6 (corresponding RNA sequences in SEQ ID NOs: 29-31, respectively), and a CMV promoter to drive expression of an S. aureus Cas9 encoded by SEQ ID NO: 10; or left and right AAV2 ITR sequences, a first U6 promoter to drive expression of a first guide RNA having a sequence selected from SEQ ID NOS: 7 and 8 and/or comprising a targeting domain sequence according to one of SEQ ID NOS: 1-3, a second U6 promoter to drive expression of a second guide RNA comprising a sequence selected from SEQ ID NOS: 7 and 8 and/or comprising a targeting domain sequence according to one of SEQ ID NOS: 4-6, and an hGRK promoter to drive expression of an S. aureus Cas9 encoded by SEQ ID NO: 10; or left and right AAV2 ITR sequences, a first U6 promoter to drive expression of a first guide RNA having a sequence selected from SEQ ID NOS: 7 and 8 and/or comprising a targeting domain sequence according to one of SEQ ID NOS: 1-3, a second U6 promoter to drive expression of a second guide RNA comprising a sequence selected from SEQ ID NOS: 7 and 8 and/or comprising a targeting domain sequence according to one of SEQ ID NOS: 4-6, and an EFS promoter to drive expression of an S. aureus Cas9 encoded by SEQ ID NO: 10.

In some embodiments, the nucleic acid or AAV vector shares at least 80%, 85%, 90%, 95%, 96%, 97%, 98%, 99% or greater sequence identity with one of the nucleic acids or AAV vectors recited above.

It should be noted that these sequences described above are exemplary, and can be modified in ways that do not disrupt the operating principles of elements they encode. Such modifications, some of which are discussed below, are within the scope of this disclosure. Without limiting the foregoing, skilled artisans will appreciate that the DNA, RNA or protein sequences of the elements of this disclosure may be varied in ways that do not interrupt their function, and that a variety of similar sequences that are substantially similar (e.g., greater than 90%, 95%, 96%, 97%, 98% or 99% sequence similarity, or in the case of short sequences such as gRNA targeting domains, sequences that differ by no more than 1, 2 or 3 nucleotides) can be utilized in the various systems, methods and AAV vectors described herein. Such modified sequences are within the scope of this disclosure.

The AAV genomes described above can be packaged into AAV capsids (for example, AAV5 capsids), which capsids can be included in compositions (such as pharmaceutical compositions) and/or administered to subjects. An exemplary pharmaceutical composition comprising an AAV capsid according to this disclosure can include a pharmaceutically acceptable carrier such as balanced saline solution (BSS) and one or more surfactants (e.g., Tween 20) and/or a thermosensitive or reverse-thermosensitive polymer (e.g., pluronic). Other pharmaceutical formulation elements known in the art may also be suitable for use in the compositions described here.

Compositions comprising AAV vectors according to this disclosure can be administered to subjects by any suitable means, including without limitation injection, for example, subretinal injection. The concentration of AAV vector within the composition is selected to ensure, among other things, that a sufficient AAV dose is administered to the retina of the subject, taking account of dead volume within the injection apparatus and the relatively limited volume that can be safely administered to the retina. Suitable doses may include, for example, 1×10¹¹ viral genomes (vg)/mL, 2×10¹¹ viral genomes (vg)/mL, 3×10¹¹ viral genomes (vg)/mL, 4×10¹¹ viral genomes (vg)/mL, 5×10¹¹ viral genomes (vg)/mL, 6×10¹¹ viral genomes (vg)/mL, 7×10¹¹ viral genomes (vg)/mL, 8×10¹¹ viral genomes (vg)/mL, 9×10¹¹ viral genomes (vg)/mL, 1×10¹² vg/mL, 2×10¹² viral genomes (vg)/mL, 3×10¹² viral genomes (vg)/mL, 4×10¹² viral genomes (vg)/mL, 5×10¹² viral genomes (vg)/mL, 6×10¹² viral genomes (vg)/mL, 7×10¹² viral genomes (vg)/mL, 8×10¹² viral genomes (vg)/mL, 9×10¹² viral genomes (vg)/mL, 1×10¹³ vg/mL, 2×10¹³ viral genomes (vg)/mL, 3×10¹³ viral genomes (vg)/mL, 4×10¹³ viral genomes (vg)/mL, 5×10¹³ viral genomes (vg)/mL, 6×10¹³ viral genomes (vg)/mL, 7×10¹³ viral genomes (vg)/mL, 8×10¹³ viral genomes (vg)/mL, or 9×10¹³ viral genomes (vg)/mL. Any suitable volume of the composition may be delivered to the subretinal space. In some instances, the volume is selected to form a bleb in the subretinal space, for example 1 microliter, 10 microliters, 50 microliters, 100 microliters, 150 microliters, 200 microliters, 250 microliters, 300 microliters, etc.

Any region of the retina may be targeted, though the fovea (which extends approximately 1 degree out from the center of the eye) may be preferred in certain instances due to its role in central visual acuity and the relatively high concentration of cone photoreceptors there relative to peripheral regions of the retina. Alternatively or additionally, injections may be targeted to parafoveal regions (extending between approximately 2 and 10 degrees off center), which are characterized by the presence of all three types of retinal photoreceptor cells. In addition, injections into the parafoveal region may be made at comparatively acute angles using needle paths that cross the midline of the retina. For instance, injection paths may extend from the nasal aspect of the sclera near the limbus through the vitreal chamber and into the parafoveal retina on the temporal side, from the temporal aspect of the sclera to the parafoveal retina on the nasal side, from a portion of the sclera located superior to the cornea to an inferior parafoveal position, and/or from an inferior portion of the sclera to a superior parafoveal position. The use of relatively small angles of injection relative to the retinal surface may advantageously reduce or limit the potential for spillover of vector from the bleb into the vitreous body and, consequently, reduce the loss of the vector during delivery. In other cases, the macula (inclusive of the fovea) can be targeted, and in other cases, additional retinal regions can be targeted, or can receive spillover doses.

For pre-clinical development purposes, systems, compositions, nucleotides and vectors according to this disclosure can be evaluated ex vivo using a retinal explant system, or in vivo using an animal model such as a mouse, rabbit, pig, nonhuman primate, etc. Retinal explants are optionally maintained on a support matrix, and AAV vectors can be delivered by injection into the space between the photoreceptor layer and the support matrix, to mimic subretinal injection. Tissue for retinal explantation can be obtained from human or animal subjects, for example mouse.

Explants are particularly useful for studying the expression of gRNAs and/or Cas9 following viral transduction, and for studying genome editing over comparatively short intervals. These models also permit higher throughput than may be possible in animal models, and can be predictive of expression and genome editing in animal models and subjects. Small (mouse, rat) and large animal models (such as rabbit, pig, nonhuman primate) can be used for pharmacological and/or toxicological studies and for testing the systems, nucleotides, vectors and compositions of this disclosure under conditions and at volumes that approximate those that will be used in clinic. Because model systems are selected to recapitulate relevant aspects of human anatomy and/or physiology, the data obtained in these systems will generally (though not necessarily) be predictive of the behavior of AAV vectors and compositions according to this disclosure in human and animal subjects.

While the foregoing exemplary embodiments have focused on guide RNAs, nucleic acids and AAV vectors targeted to the CEP290 gene, it will be appreciated by those of skill in the art that the nucleic acids and vectors of this disclosure may be used in the editing of other gene targets and the treatment of other diseases such as hereditary retinopathies that may be treated by editing of genes other than CEP290. FIGS. 1B and 1C illustrate two exemplary AAV vectors that may be used to transduce retinal cells, including without limitation retinal photoreceptor cells such as rod photoreceptors and/or cone photoreceptors, and/or other retinal cell types. The AAV genome of FIG. 1B comprises two guide RNAs according to SEQ ID NOS: 7 and/or 8, and a promoter sequence according to one of SEQ ID NOS: 13-15 driving expression of an S. aureus Cas9 comprising one or two nuclear localization signals and, optionally, a polyadenylation signal. The vector may additionally include ITRs such as AAV2 ITRs, or other sequences that may be selected for the specific application to which the vector will be employed. As is shown in FIG. 1C, other vectors within the scope of this disclosure may include only 1 guide RNA. Thus, in specific embodiments, an AAV genome of this disclosure may encode a CMV promoter for the Cas9 and one guide RNA having a sequence comprising, or sharing at least 90% sequence identity with, a sequence selected from SEQ ID NOS: 7 and 8; a CMV promoter for the Cas9 and two guide RNAs, each having a sequence comprising, or sharing at least 90% sequence identity with, a sequence selected from SEQ ID NOS: 7 and 8; an hGRK promoter for the Cas9 and one guide RNA having a sequence comprising, or sharing at least 90% sequence identity with, a sequence selected from SEQ ID NOS: 7 and 8; an hGRK promoter for the Cas9 and two guide RNAs, each having a sequence comprising, or sharing at least 90% sequence identity with, a sequence selected from SEQ ID NOS: 7 and 8; an EFS promoter for the Cas9 and one guide RNA having a sequence comprising, or sharing at least 90% sequence identity with, a sequence selected from SEQ ID NOS: 7 and 8; an EFS promoter for the Cas9 and two guide RNAs, each having a sequence comprising, or sharing at least 90% sequence identity with, a sequence selected from SEQ ID NOS: 7 and 8.

### Genome Editing Systems

The term “genome editing system” refers to any system having RNA-guided DNA editing activity. Genome editing systems of the present disclosure include at least two components adapted from naturally occurring CRISPR systems: a gRNA and an RNA-guided nuclease. These two components form a complex that is capable of associating with a specific nucleic acid sequence in a cell and editing the DNA in or around that nucleic acid sequence, for example by making one or more of a single-strand break (an SSB or nick), a double-strand break (a DSB) and/or a base substitution.

Naturally occurring CRISPR systems are organized evolutionarily into two classes and five types (Makarova 2011, incorporated by reference herein), and while genome editing systems of the present disclosure may adapt components of any type or class of naturally occurring CRISPR system, the embodiments presented herein are generally adapted from Class 2, and type II or V CRISPR systems. Class 2 systems, which encompass types II and V, are characterized by relatively large, multidomain RNA-guided nuclease proteins (e.g., Cas9 or Cpf1) that form ribonucleoprotein (RNP) complexes with gRNAs. gRNAs, which are discussed in greater detail below, can include single crRNAs in the case of Cpf1 or duplexed crRNAs and tracrRNAs in the case of Cas9. RNP complexes, in turn, associate with (i.e., target) and cleave specific loci complementary to a targeting (or spacer) sequence of the crRNA. Genome editing systems according to the present disclosure similarly target and edit cellular DNA sequences. but differ significantly from CRISPR systems occurring in nature. For example, the unimolecular gRNAs described herein do not occur in nature, and both gRNAs and RNA-guided nucleases according to this disclosure can incorporate any number of non-naturally occurring modifications.

Genome editing systems can be implemented in a variety of ways, and different implementations may be suitable for any particular application. For example, a genome editing system is implemented, in certain embodiments, as a protein/RNA complex (a ribonucleoprotein, or RNP), which can be included in a pharmaceutical composition that optionally includes a pharmaceutically acceptable carrier and/or an encapsulating agent, such as a lipid or polymer micro- or nano-particle, micelle, liposome, etc. In other embodiments, a genome editing system is implemented as one or more nucleic acids encoding the RNA-guided nuclease and gRNA components described above (optionally with one or more additional components); in still other embodiments, the genome editing system is implemented as one or more vectors comprising such nucleic acids, for example a viral vector such as an AAV; and in still other embodiments, the genome editing system is implemented as a combination of any of the foregoing. Additional or modified implementations that operate according to the principles set forth herein will be apparent to the skilled artisan and are within the scope of this disclosure.

It should be noted that the genome editing systems of the present invention can be targeted to a single specific nucleotide sequence, or can be targeted to—and capable of editing in parallel—two or more specific nucleotide sequences through the use of two or more gRNAs. The use of two or more gRNAs targeted to different sites is referred to as “multiplexing” throughout this disclosure, and can be employed to target multiple, unrelated target sequences of interest, or to form multiple SSBs and/or DSBs within a single target domain and, in some cases, to generate specific edits within such target domain. For example, this disclosure and Maeder both describe a genome editing system for correcting a point mutation (C.2991+1655A to G) in the human CEP290 gene that results in the creation of a cryptic splice site, which in turn reduces or eliminates the function of the gene. The genome editing system of Maeder utilizes two gRNAs targeted to sequences on either side of (i.e., flanking) the point mutation, and forms DSBs that flank the mutation. This, in turn, promotes deletion of the intervening sequence, including the mutation, thereby eliminating the cryptic splice site and restoring normal gene function.

As another example, International Patent Publication No. WO2016/073990 by Cotta-Ramusino et al. (“Cotta-Ramusino”), incorporated by reference herein, describes a genome editing system that utilizes two gRNAs in combination with a Cas9 nickase (a Cas9 that makes a single strand nick such as S. pyogenes D10A), an arrangement termed a “dual-nickase system.” The dual-nickase system of Cotta-Ramusino is configured to make two nicks on opposite strands of a sequence of interest that are offset by one or more nucleotides, which nicks combine to create a double strand break having an overhang (5′ in the case of Cotta-Ramusino, though 3′ overhangs are also possible). The overhang, in turn, can facilitate homology directed repair events in some circumstances. And, as another example, International Patent Publication No. WO2015/070083 by Zhang et al., incorporated by reference herein, describes a gRNA targeted to a nucleotide sequence encoding Cas9 (referred to as a “governing” gRNA), which can be included in a genome editing system comprising one or more additional gRNAs to permit transient expression of a Cas9 that might otherwise be constitutively expressed, for example in some virally transduced cells. These multiplexing applications are intended to be exemplary, rather than limiting, and the skilled artisan will appreciate that other applications of multiplexing are generally compatible with the genome editing systems described here.

Genome editing systems can, in some instances, form double strand breaks that are repaired by cellular DNA double-strand break mechanisms such as non-homologous end joining (NHEJ), or homology directed repair (HDR). These mechanisms are described throughout the literature (see, e.g., Davis 2014 (describing Alt-HDR), Frit 2014 (describing Alt-NHEJ), and Iyama 2013 (describing canonical HDR and NHEJ pathways generally), all of which are incorporated by reference herein).

Where genome editing systems operate by forming DSBs, such systems optionally include one or more components that promote or facilitate a particular mode of double-strand break repair or a particular repair outcome. For example, Cotta-Ramusino also describes genome editing systems in which a single stranded oligonucleotide “donor template” is added; the donor template is incorporated into a target region of cellular DNA that is cleaved by the genome editing system, and can result in a change in the target sequence.

In other cases, genome editing systems modify a target sequence, or modify expression of a gene in or near the target sequence, without causing single- or double-strand breaks. For example, a genome editing system can include an RNA-guided nuclease/cytidine deaminase fusion protein, and can operate by generating targeted C-to-A substitutions. Suitable nuclease/deaminase fusions are described in Komor 2016, which is incorporated by reference. Alternatively, a genome editing system can utilize a cleavage-inactivated (i.e., a “dead”) nuclease, such as a dead Cas9, and can operate by forming stable complexes on one or more targeted regions of cellular DNA, thereby interfering with functions involving the targeted region(s) such as mRNA transcription and chromatin remodeling.

Guide RNA (gRNA)

The terms guide RNA and gRNA refer to any nucleic acid that promotes the specific association (or “targeting”) of an RNA-guided nuclease such as a Cas9 or a Cpf1 to a target sequence such as a genomic or episomal sequence in a cell. gRNAs can be unimolecular (comprising a single RNA molecule, and referred to alternatively as chimeric), or modular (comprising more than one, and typically two, separate RNA molecules, such as a crRNA and a tracrRNA, which are usually associated with one another, for example by duplexing). gRNAs and their component parts are described throughout the literature (see, e.g., Briner 2014, which is incorporated by reference; see also Cotta-Ramusino).

In bacteria and archea, type II CRISPR systems generally comprise an RNA-guided nuclease protein such as Cas9, a CRISPR RNA (crRNA) that includes a 5′ region that is complementary to a foreign sequence, and a trans-activating crRNA (tracrRNA) that includes a 5′ region that is complementary to, and forms a duplex with, a 3′ region of the crRNA. While not intending to be bound by any theory, it is thought that this duplex facilitates the formation of—and is necessary for the activity of—the Cas9/gRNA complex. As type II CRISPR systems were adapted for use in gene editing, it was discovered that the crRNA and tracrRNA could be joined into a single unimolecular or chimeric gRNA, for example by means of a four nucleotide (e.g., GAAA) “tetraloop” or “linker” sequence bridging complementary regions of the crRNA (at its 3′ end) and the tracrRNA (at its 5′ end) (Mali 2013; Jiang 2013; Jinek 2012; all incorporated by reference herein).

gRNAs, whether unimolecular or modular, include a targeting domain that is fully or partially complementary to a target domain within a target sequence, such as a DNA sequence in the genome of a cell where editing is desired. In certain embodiments, this target sequence encompasses or is proximal to a CEP290 target position. Targeting domains are referred to by various names in the literature, including without limitation “guide sequences” (Hsu 2013, incorporated by reference herein), “complementarity regions” (Cotta-Ramusino), “spacers” (Briner 2014), and generically as “crRNAs” (Jiang 2013). Irrespective of the names they are given, targeting domains are typically 10-30 nucleotides in length, preferably 16-24 nucleotides in length (for example, 16, 17, 18, 19, 20, 21, 22, 23 or 24 nucleotides in length), and are at or near the 5′ terminus of in the case of a Cas9 gRNA, and at or near the 3′ terminus in the case of a Cpf1 gRNA.

In addition to the targeting domains, gRNAs typically (but not necessarily, as discussed below) include a plurality of domains that influence the formation or activity of gRNA/Cas9 complexes. For example, as mentioned above, the duplexed structure formed by first and secondary complementarity domains of a gRNA (also referred to as a repeat:anti-repeat duplex) interacts with the recognition (REC) lobe of Cas9 and may mediate the formation of Cas9/gRNA complexes (Nishimasu 2014; Nishimasu 2015; both incorporated by reference herein). It should be noted that the first and/or second complementarity domains can contain one or more poly-A tracts, which can be recognized by RNA polymerases as a termination signal. The sequence of the first and second complementarity domains are, therefore, optionally modified to eliminate these tracts and promote the complete in vitro transcription of gRNAs, for example through the use of A-G swaps as described in Briner 2014, or A-U swaps. These and other similar modifications to the first and second complementarity domains are within the scope of the present disclosure.

Along with the first and second complementarity domains, Cas9 gRNAs typically include two or more additional duplexed regions that are necessary for nuclease activity in vivo but not necessarily in vitro (Nishimasu 2015). A first stem-loop near the 3′ portion of the second complementarity domain is referred to variously as the “proximal domain,” (Cotta-Ramusino) “stem loop 1” (Nishimasu 2014; Nishimasu 2015) and the “nexus” (Briner 2014). One or more additional stem loop structures are generally present near the 3′ end of the gRNA, with the number varying by species: S. pyogenes gRNAs typically include two 3′ stem loops (for a total of four stem loop structures including the repeat:anti-repeat duplex), while s. aureus and other species have only one (for a total of three). A description of conserved stem loop structures (and gRNA structures more generally) organized by species is provided in Briner 2014.

Skilled artisans will appreciate that gRNAs can be modified in a number of ways, some of which are described below, and these modifications are within the scope of disclosure. For economy of presentation in this disclosure, gRNAs may be presented by reference solely to their targeting domain sequences.

gRNA Modifications

The activity, stability, or other characteristics of gRNAs can be altered through the incorporation of chemical and/or sequential modifications. As one example, transiently expressed or delivered nucleic acids can be prone to degradation by, e.g., cellular nucleases. Accordingly, the gRNAs described herein can contain one or more modified nucleosides or nucleotides which introduce stability toward nucleases. While not wishing to be bound by theory it is also believed that certain modified gRNAs described herein can exhibit a reduced innate immune response when introduced into a population of cells, particularly the cells of the present invention. As noted above, the term “innate immune response” includes a cellular response to exogenous nucleic acids, including single stranded nucleic acids, generally of viral or bacterial origin, which involves the induction of cytokine expression and release, particularly the interferons, and cell death.

One common 3′ end modification is the addition of a poly A tract comprising one or more (and typically 5-200) adenine (A) residues. The poly A tract can be contained in the nucleic acid sequence encoding the gRNA, or can be added to the gRNA during chemical synthesis, or following in vitro transcription using a polyadenosine polymerase (e.g., E. coli Poly(A)Polymerase). In vivo, poly-A tracts can be added to sequences transcribed from DNA vectors through the use of polyadenylation signals. Examples of such signals are provided in Maeder.

### RNA-Guided Nucleases

RNA-guided nucleases according to the present disclosure include, without limitation, naturally-occurring Class 2 CRISPR nucleases such as Cas9, and Cpf1, as well as other nucleases derived or obtained therefrom. In functional terms, RNA-guided nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g., Cas9 vs. Cpf1), species (e.g., S. pyogenes vs. S. aureus) or variation (e.g., full-length vs. truncated or split; naturally-occurring PAM specificity vs. engineered PAM specificity).

Turning to the PAM sequence, this structure takes its name from its sequential relationship to the “protospacer” sequence that is complementary to gRNA targeting domains (or “spacers”). Together with protospacer sequences, PAM sequences define target regions or sequences for specific RNA-guided nuclease/gRNA combinations.

Various RNA-guided nucleases may require different sequential relationships between PAMs and protospacers. In general, Cas9s recognize PAM sequences that are 5′ of the protospacer as visualized relative to the top or complementary strand.

In addition to recognizing specific sequential orientations of PAMs and protospacers, RNA-guided nucleases generally recognize specific PAM sequences. S. aureus Cas9, for example, recognizes a PAM sequence of NNGRRT, wherein the N sequences are immediately 3′ of the region recognized by the gRNA targeting domain. S. pyogenes Cas9 recognizes NGG PAM sequences. It should also be noted that engineered RNA-guided nucleases can have PAM specificities that differ from the PAM specificities of similar nucleases (such as the naturally occurring variant from which an RNA-guided nuclease is derived, or the naturally occurring variant having the greatest amino acid sequence homology to an engineered RNA-guided nuclease). Modified Cas9s that recognize alternate PAM sequences are described below.

RNA-guided nucleases are also characterized by their DNA cleavage activity: naturally-occurring RNA-guided nucleases typically form DSBs in target nucleic acids, but engineered variants have been produced that generate only SSBs (discussed above; see also Ran 2013, incorporated by reference herein), or that do not cut at all.

### Cas9

Crystal structures have been determined for S. pyogenes Cas9 (Jinek 2014), and for S. aureus Cas9 in complex with a unimolecular gRNA and a target DNA (Nishimasu 2014; Anders 2014; and Nishimasu 2015).

A naturally occurring Cas9 protein comprises two lobes: a recognition (REC) lobe and a nuclease (NUC) lobe; each of which comprise particular structural and/or functional domains. The REC lobe comprises an arginine-rich bridge helix (BH) domain, and at least one REC domain (e.g., a REC1 domain and, optionally, a REC2 domain) The REC lobe does not share structural similarity with other known proteins, indicating that it is a unique functional domain. While not wishing to be bound by any theory, mutational analyses suggest specific functional roles for the BH and REC domains: the BH domain appears to play a role in gRNA:DNA recognition, while the REC domain is thought to interact with the repeat:anti-repeat duplex of the gRNA and to mediate the formation of the Cas9/gRNA complex.

The NUC lobe comprises a RuvC domain, an HNH domain, and a PAM-interacting (PI) domain. The RuvC domain shares structural similarity to retroviral integrase superfamily members and cleaves the non-complementary (i.e., bottom) strand of the target nucleic acid. It may be formed from two or more split RuvC motifs (such as RuvC I, RuvCII, and RuvCIII in S. pyogenes and S. aureus). The HNH domain, meanwhile, is structurally similar to HNH endonuclease motifs, and cleaves the complementary (i.e., top) strand of the target nucleic acid. The PI domain contributes to PAM specificity.

### Modifications of RNA-Guided Nucleases

The RNA-guided nucleases described above have activities and properties that are useful in a variety of applications, but the skilled artisan will appreciate that RNA-guided nucleases may also be modified in certain instances, to alter cleavage activity, PAM specificity, or other structural or functional features.

Turning first to modifications that alter cleavage activity, mutations that reduce or eliminate the activity of domains within the NUC lobe have been described above. Exemplary mutations that may be made in the RuvC domains, in the Cas9 HNH domain, or in the Cpf1 Nuc domain are described in Ran and Yamano, as well as in Cotta-Ramusino. In general, mutations that reduce or eliminate activity in one of the two nuclease domains result in RNA-guided nucleases with nickase activity, but it should be noted that the type of nickase activity varies depending on which domain is inactivated. As one example, inactivation of a RuvC domain of a Cas9 will result in a nickase that cleaves the complementary strand, while inactivation of a Cas9 HNH domain results in a nickase that cleaves the non-complementary strand.

Modifications of PAM specificity relative to naturally occurring Cas9 reference molecules has been described for both S. pyogenes (Kleinstiver 2015a) and S. aureus (Kleinstiver 2015b). Modifications that improve the targeting fidelity of Cas9 have also been described (Kleinstiver 2016). Each of these references is incorporated by reference herein.

RNA-guided nucleases have been split into two or more parts (see, e.g., Zetsche 2015; Fine 2015; both incorporated by reference).

RNA-guided nucleases are, in some cases, size-optimized or truncated, for example via one or more deletions that reduce the size of the nuclease while still retaining gRNA association, target and PAM recognition, and cleavage activities. In certain embodiments, RNA guided nucleases are bound, covalently or non-covalently, to another polypeptide, nucleotide, or other structure, optionally by means of a linker. RNA-guided nucleases also optionally include a tag, such as a nuclear localization signal to facilitate movement of RNA-guided nuclease protein into the nucleus.

The foregoing list of modifications is intended to be exemplary in nature, and the skilled artisan will appreciate that other modifications may be possible or desirable in certain applications. For brevity, therefore, certain systems, methods and compositions of the present disclosure are exemplified by reference to particular RNA-guided nucleases, but it should be understood that the RNA-guided nucleases used may be modified in ways that do not alter the exemplified operating principles. Such modifications are within the scope of the present disclosure.

### EXAMPLES

The following Examples are illustrative and are not intended to limit the scope or content of the invention in any way.

### Example 1: AAV Transduction of Genome Editing Systems in Mouse Retinal Explants

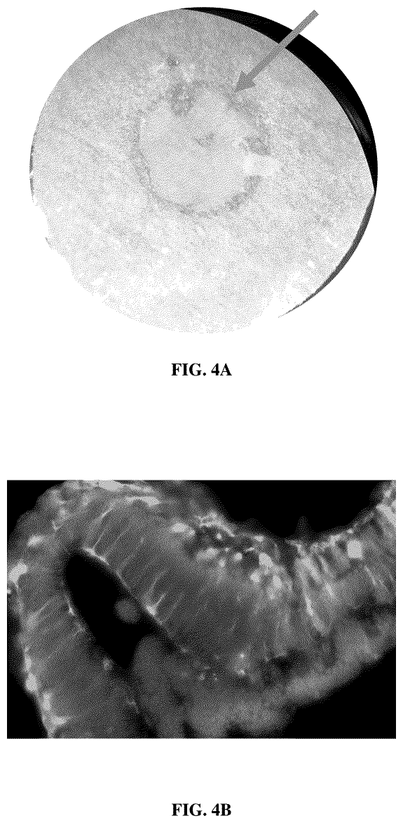

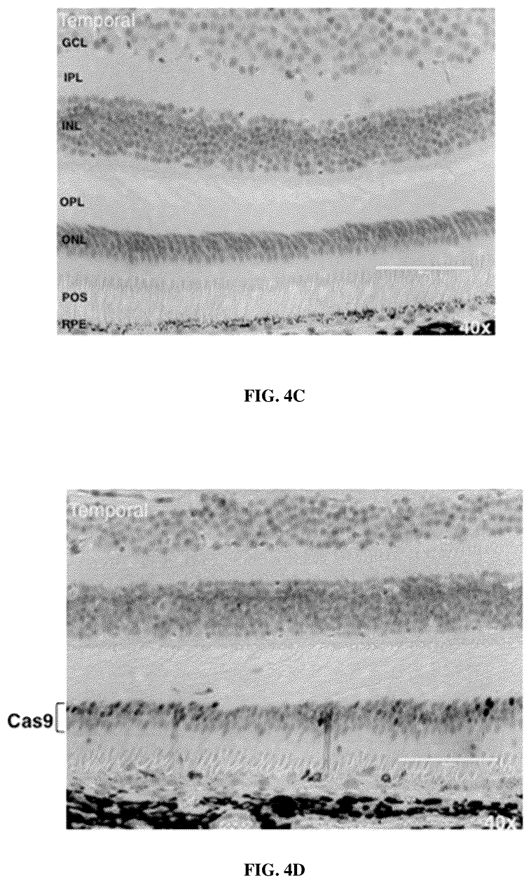

To assess the ability of the AAV vectors described above to transduce CRISPR/Cas9 genome editing systems into retinal cells in situ, an ex vivo explant system was developed. FIG. 4A shows a representative image of an explanted mouse retina on a support matrix, with the tissue indicated by the gray arrow. Explants were harvested at 7- or 10-day time points, and histological, DNA, RNA and/or protein samples were produced. FIG. 4B shows a representative fluorescence micrograph from a retinal explant treated with an AAV vector carrying a GFP reporter, demonstrating successful transduction of an AAV payload in cells in multiple layers of the retina.

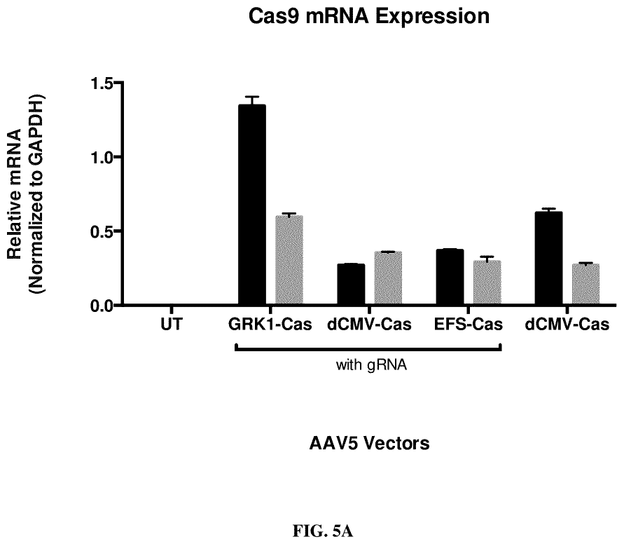

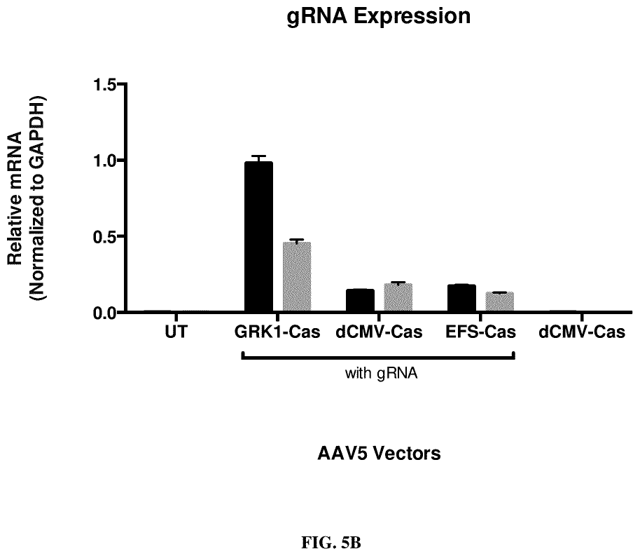

mRNA samples taken from retinal explants further demonstrate that genome editing systems according to the present disclosure are effectively transduced by these AAV vectors: FIG. 5A and FIG. 5B show expression of Cas9 mRNA and gRNA, respectively, normalized to the expression of GAPDH. As expected, untreated samples did not express Cas9 or gRNA, and gRNA was not detected in samples that were not transduced with gRNA coding sequences. Cas9 expression was observed in three AAV constructs in which Cas9 expression was driven by hGRK1, CMV or EFS promoters. The observation of Cas9 mRNA and gRNA in samples transduced with vectors in which Cas9 expression is driven by the retinal photoreceptor cell specific hGRK1 promoter indicates that these vectors can transduce genome editing systems in photoreceptor cells in situ.

DNA samples from retinal explants treated with AAV vectors were sequenced, and indel species were identified. The AAV vectors used in the mouse explant system included guides with targeting domains specific to the mouse CEP290 gene but targeted to the same region of intron 26 as the human guides presented above; aside from the specific guide sequences used, the AAV vectors used were the same as those described above. Table 7 shows a wild type (WT) mouse sequence, with left and right guide sequences italicized, and three representative indels of +1, -4 and -246 aligned with the WT sequence. In the table, three periods ( . . . ) represent an abbreviation of the sequence read for ease of presentation, while dashes (-) represent alignment gaps and underlined nucleotides represent insertions. Insofar as DNA sequencing of explants treated with AAV vectors utilizing the photoreceptor specific hGRK1 promoter revealed indel formation, these data demonstrate genome editing of a CEP290 target site in retinal photoreceptors.

TABLE 7
Representative Indels in Mouse Retinal Explants

| | |
|---|---|
| WT | CCCTCAAACACATGTCTCACGCAGCTTAGACATTCT . . . CAGAACTCGGTCAG- |
| | CATGCTACAGATAGCTTATCT |
| | (SEQ ID NO: 18) (SEQ ID NO: 19) |
| +1 | CCCTCAAACACATGTCTCACGCAGCTTAGACATTCT . . . CAGAACTCGGTCAGGCATGCTACAGATAGCTTATC |
| | T |
| | (SEQ ID NO: 18) (SEQ ID NO: 34) |
| −4 | CCCTCAAACACATGTCTCACGCAGCTTAGACATTCT . . . CAGAACTCGG----- |
| | CATGCTACAGATAGCTTATCT |
| | (SEQ ID NO: 18) (SEQ ID NO: 35) |
| −246 | CCCTCAAAG--------------------------- . . . --------------- |
| | CATGCTACAGATAGCTTATCT |
| | (SEQ ID NO: 36) (SEQ ID NO: 37) |

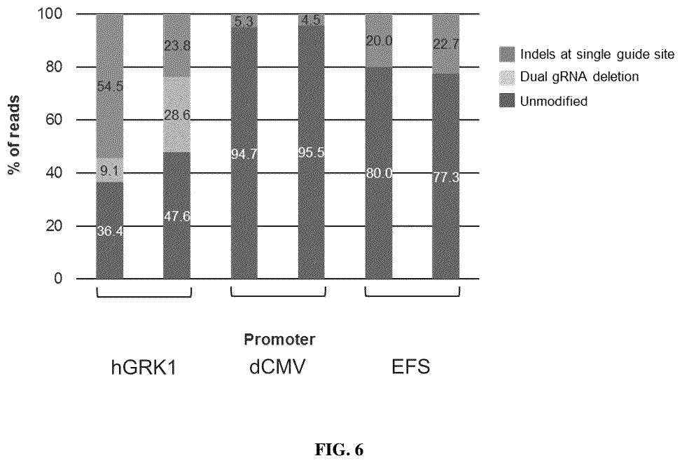

FIG. 6 summarizes the estimated frequencies of particular editing events in individual mouse explants transduced with AAV vectors according to the present disclosure. In samples transduced with AAV vectors in which Cas9 expression was driven by the hGRK1 promoter, deletions of sequences between gRNAs (guide sites) were consistently observed, as were indels at one of the two guide sites. Indels at one of the two guide sites were also observed in explants transduced with CMV and EFS vectors.

Taken together, these results demonstrate the transduction of CRISPR/Cas9 genome editing systems into cells, including photoreceptor cells, in the intact mouse retina and the editing (including deletion) of a CEP290 target site in retinal photoreceptors in situ.

### Example 2: AAV Transduction of Genome Editing Systems in Primate Retina In Vivo

To assess the ability of the AAV vectors described above to transduce CRISPR/Cas9 genome editing systems into retinal cells in vivo, a primate subretinal injection procedure was developed. Cynomolgus macaques received a bilateral subretinal injections of an AAV5 vector encoding S. aureus Cas9 operably linked to an EFS, CMV or hGRK promoter sequence, and gRNAs C1 and C2, targeted to an intronic region of the cynomolgus CEP290 gene and comprising targeting sequences as set forth in Table 8. AAV injections were given at dosages of 4×10¹⁰ (low) or 4×10¹¹ (high) viral genomes (vg). Experimental conditions are summarized in Table 9.

TABLE 8
Cynomolgus gRNA Targeting Domain Sequences

| | Targeting Domain | Targeting Domain |
|---|---|---|
| gRNA | Sequence (DNA) | Sequence (RNA) |
| C1 | GGCCGGCTAATTTAGTAGAGA | GGCCGGCUAAUUUAGUAGAGA |
| | (SEQ ID NO: 20) | (SEQ ID NO: 38) |
| C2 | GTTATGAAGAATAATACAAA | GUUAUGAAGAAUAAUACAAA |
| | (SEQ ID NO: 21) | (SEQ ID NO: 39) |

TABLE 9
Cynomolgus Treatment Conditions

| Group | Vector | Dose (vg/eye) |
|---|---|---|
| CMV-low | CEPgRNAs-dCMV-Cas9 | 4 × 10¹⁰ |
| CMV-high | CEPgRNAs-dCMV-Cas9 | 4 × 10¹¹ |
| EFS-low | CEPgRNAs-EFS-Cas9 | 4 × 10¹⁰ |
| EFS-high | CEPgRNAs-EFS-Cas9 | 4 × 10¹¹ |
| GRK-low | CEPgRNAs-GRK1-Cas9 | 4 × 10¹⁰ |
| GRK-high | CEPgRNAs-GRK1-Cas9 | 4 × 10¹¹ |
| Vehicle | GRK1-GFP/Vehicle | 4 × 10¹¹ |

6 or 8 mm retinal tissue punches were obtained from AAV-treated and Vehicle-treated retinas at 6 and 13 weeks post injection, and genomic DNA was harvested. Sequencing was performed by using a proprietary methodology (Uni-Directional Targeted Sequencing, or UDiTaS) described in commonly assigned, copending U.S. Provisional Patent Application No. 62/443,212, which is incorporated by reference herein in its entirety. Data from two UDiTaS sequencing reactions with individual upstream or downstream primers was combined by assuming complete overlap of indels at the two different gRNA cut sites and by averaging the rates of inversions and deletions observed in the two sequencing reactions.

Histological analysis demonstrated successful transduction of primate photoreceptor cells using genome editing systems as disclosed herein. FIG. 4C depicts Cas9 antibody staining in a vehicle-control tissue punch from a primate retina, while FIG. 4D shows Cas9 expression in a punch from a primate retina treated with an AAV5 vector encoding S. aureus Cas9 operably linked to an hGRK promoter sequence. The figures show that the outer nuclear layer (ONL) in the AAV5 vector-treated punch contains Cas9 protein, while the ONL from the vehicle control punch does not. This demonstrates successful transduction of cells in this layer. No detectable Cas9 expression was detected in cells outside the ONL. Because the hGRK promoter is photoreceptor specific, these data indicate that the systems and methods of this disclosure result in Cas9 expression among retinal photoreceptor cells in primates.

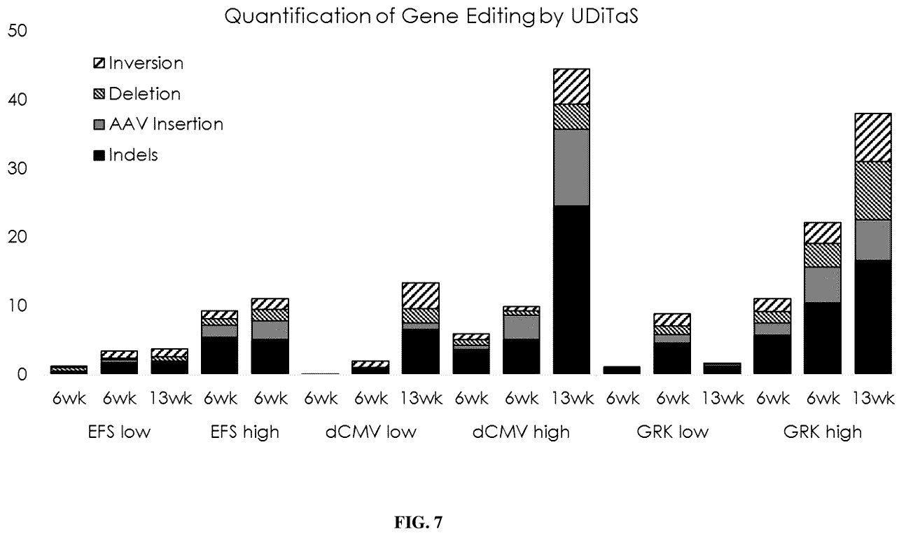

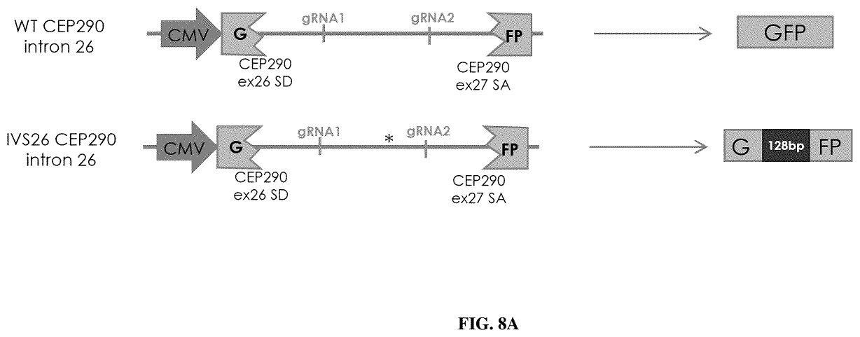

FIG. 7 shows the frequency with which specific edits (indels, insertions, deletions and inversions, were observed in each condition. In both the CMV-high and GRK-high conditions, the frequency of editing events approached or exceeded 40% of reads at the 13-week timepoints. Frequencies of specific edits observed in each experimental condition at each timepoint are listed in Table 10, below. 13 weeks timepoints for the EFS-high condition were not obtained.

TABLE 10
Editing Frequencies Observed in Cynomolgus
Treatment Conditions at 6 and 13 Weeks

| | | Total | | | | |
|---|---|---|---|---|---|---|
| | | editing | Inversions | Deletions | Insertions | Indels |
| EFS- | 6 week | 2.4% | 0.6% | 0.4% | 0.3% | 1.1% |
| low | 13 week | 3.8% | 1.2% | 0.6% | 0.0% | 2.0% |
| EFS- | 6 week | 10.2% | 1.4% | 1.3% | 2.3% | 5.3% |
| high | 13 week | — | — | — | — | — |
| CMV- | 6 week | 1.1% | 0.5% | 0.0% | 0.1% | 0.5% |
| low | 13 week | 13.4% | 3.7% | 2.1% | 0.9% | 6.6% |
| CMV- | 6 week | 8.0% | 0.7% | 0.7% | 2.1% | 4.4% |
| high | 13 week | 44.5% | 5.1% | 3.7% | 11.2% | 24.5% |
| GRK- | 6 week | 5.0% | 0.9% | 0.7% | 0.7% | 2.7% |
| low | 13 week | 1.6% | 0.0% | 0.0% | 0.3% | 1.3% |
| GRK- | 6 week | 16.6% | 2.5% | 2.5% | 3.5% | 8.1% |
| high | 13 week | 38.0% | 7.0% | 8.5% | 5.9% | 16.7% |

It should be noted that the hGRK1 promoter is photoreceptor specific, and that the genome editing system encoded by the AAV5 vector would only be functional in photoreceptor cells. It is reasonable to conclude, therefore, that the percentages of reads obtained from tissue punches, which include other retinal cell types, are lower than the percentages that would be observed in photoreceptor cells alone. Together, these data demonstrate transduction of a CRISPR/Cas9 system into a primate retina by subretinal injection of AAV, in vivo, and the generation of targeted alterations in a CEP290 gene sequence in primate photoreceptor cells in vivo.

### Example 3: Correction of IVS26 Splicing Defect by Inversions and Deletions

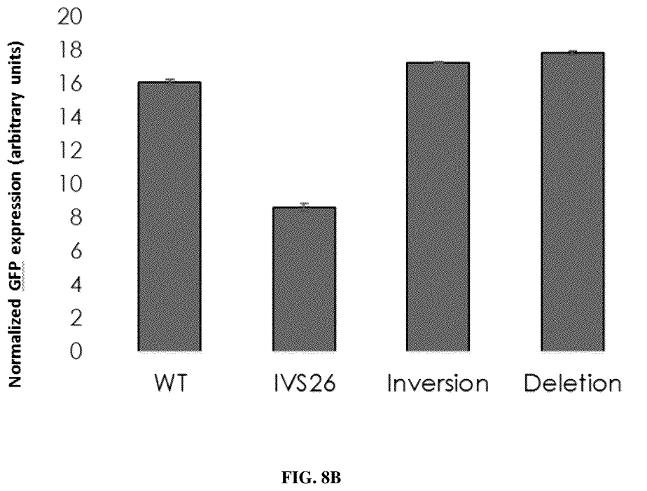

To verify that deletions and inversions of the intronic region including the IVS26 mutation correct the splicing defect observed in CEP290 associated disease, a reporter assay was developed utilizing four reporter constructs having the general design depicted in FIG. 8A pAD26_SplitGFP+WildType_CEP290_Kan (SEQ ID NO:22); pAD27_SplitGFP+Mutant_CEP290_Kan (SEQ ID NO:23); pAD28_SplitGFP+Mutant_CEP290_Inverted_Kan (SEQ ID NO:24); and pAD29_SplitGFP+DeletionCEP290_Kan (SEQ ID NO:25). These constructs were transfected into U2OS cells at the concentrations shown in FIG. 8B, and GFP and mCherry expression was quantitated for each condition across three bioreplicates. Each of the four reporter constructs included a sequence encoding a split-green-fluorescent protein (GFP) reporter gene incorporating a 2217 bp human CEP 290 intron sequence corresponding to (a) wild type (WT), (b) the IVS26 mutation, (c) a deletion of the intronic sequence between two human CEP290 target sites, including the IVS26 mutation and the cryptic exon observed in mRNAs from subjects with CEP290 associated disease, as would result from the use of a genome editing system according to the present disclosure, or (d) an inversion of the intronic sequence between the two human CEP290 target sites, including the IVS26 mutation and the cryptic exon as would result from the use of a genome editing system of this disclosure. The construct is designed such that correct splicing is necessary for GFP expression. Thus, the presence of the cryptic splice acceptor site in the IVS26 condition, but not the WT condition, will result in disrupted GFP transcripts encoding non-functional GFP proteins; modifications at CEP290 target sites that result in the removal or alteration of the IVS26 mutation would rescue the expression of functional GFP protein. As shown in FIG. 8B, functional GFP protein is expressed at a high baseline level in cells treated with the WT construct, expression is reduced in the IVS26 condition, and is returned to the WT baseline level in the deletion and inversion conditions. These data indicate that the aberrant mRNA splicing caused by the IVS26 mutation is rescued by either deletion or inversion of the intronic sequence comprising that mutation.

### Example 4: AAV5 Transduction of Genome Editing Systems in Human Retinal Explants

To further establish that the genome editing systems of the present disclosure supported targeted gene editing in human retinal cells, e.g., fully mature human photoreceptors in situ, an ex vivo human retina explant system was developed. Purified AAV5 vectors were selected that encoded S. aureus Cas9 operably linked to an hGRK1 or CMV promoter sequence and first and second gRNAs comprising targeting sequences according to SEQ ID NOS: 1 and 4, respectively, and backbone sequences according to SEQ ID NO: 8. As discussed above, these guides are targeted to the intronic region of the CEP290 gene on opposite sides of the IVS26 A>G mutation (Table 2). Human cadaver donor eyes were obtained within approximately 5 hours post-mortem and 3 mm punches were immobilized on a culture substrate as described above. Retinal explants were treated with AAV vectors at either a low dose of 1×10¹¹ vg or a high dose of 5×10¹¹ vg. Experimental conditions are summarized in Table 11.

TABLE 11
Human Treatment Conditions

| Group | Vector | Dose (vg/punch) |
|---|---|---|
| CMV-low | CEPgRNAs-dCMV-Cas9 | 1 × 10¹¹ |
| CMV-high | CEPgRNAs-dCMV-Cas9 | 5 × 10¹¹ |
| GRK-low | CEPgRNAs-GRK1-Cas9 | 1 × 10¹¹ |
| GRK-high | CEPgRNAs-GRK1-Cas9 | 5 × 10¹¹ |
| Vehicle | GRK1-GFP/Vehicle | 5 × 10¹¹ |

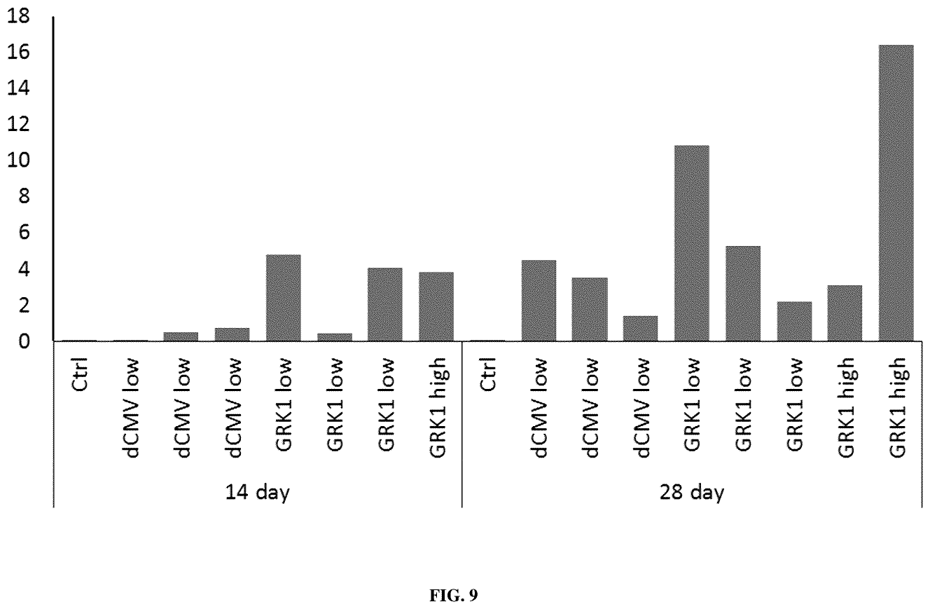

DNA samples from human retinal explants treated with AAV vectors were sequenced at either 14 or 28 days post-transduction, and inversions and deletions were identified. FIG. 9 summarizes the productive editing observed in human retinal explants 14 and 28 days after transduction with the various AAV vectors. Productive editing was defined as total edits (equal to the sum of the rates of inversions and deletions) capable of correcting the LCA10-associated splice mutation in the CEP290 gene (FIG. 9). The most productive editing was observed at 16.4% at the 28 day time point for the GRK-high condition. These data demonstrate transduction of a CRISPR/Cas9 system into a human retina by subretinal injection of AAV and the generation of targeted alterations in a CEP290 gene sequence in human photoreceptor cells in situ.

### Example 5: AAV5 Transduction of Genome Editing Systems in Live Transgenic IVS26 Knock-In Mice

To further establish that the genome editing systems of the present disclosure supported targeted gene editing of the human CEP290 target position in mature photoreceptors in vivo, an IVS26 12 KI mouse model was employed. In this model, the human CEP290 exon 26, intron 26 with the IVS26 mutation (13 c.2991+1655A>G) and exon 27 have been inserted into the murine CEP290 gene via homologous recombination. AAV5 vectors encoding (i) S. aureus Cas9 operably linked to the photoreceptor-specific hGRK1 promoter sequence, and (ii) first and second gRNAs comprising targeting sequences according to SEQ ID NOS: 1 and 4, respectively, and having gRNA backbone sequences according to SEQ ID NO: 8 were used as described in Example 4. The vectors were administered subretinally (toward the temporal side of the retina near the optic nerve) in both eyes of each animal at doses of 1×10¹¹ vg/mL, 1×10¹² vg/mL or 1×10¹³ vg/mL; a vehicle group (containing BSS with 0.014% Tween20) was also used in the study as a control. Subretinal injections were conducted in anesthetized mice in accordance with NIH animal care guidelines. For each injection, a blunt-ended needle (33-gauge, 0.5 in; Hamilton company) on a 5m1 Hamilton syringe was inserted through the scleral incision, posterior to the lens, and was advanced centrally toward the temporal retina until resistance was felt. Care was taken to avoid the damaging the lens as the cannula was advanced. A volume of 1 microliter of AAV formulation or vehicle control containing 0.2 mg/mL of fluorescein was injected into the subretinal space, forming a bleb; fluorescein was used to visualize the bleb and to confirm successful injection. Animals were euthanized at 6- and 12-week timepoints, and retinal genomic DNA and RNA were isolated for determining the gene editing efficiency (by UDiTaS) and Cas9/gRNA levels (by RT_PCR), respectively.

Experimental conditions are summarized in Table 12, along with rates of insertion and deletion from individual retinas as measured by UDiTaS.

TABLE 12
Inversion and deletion rates in IVS26 KI mouse retinas

| | Dose | | | |
|---|---|---|---|---|
| | 1 × 10¹² vg | | 1 × 10¹³ vg | |
| Timepoint | 6 weeks | 12 weeks | 6 weeks | 12 weeks |
| Inversions | 4.68% | 3.91% | 2.06% | 1.88% |
| Deletions | 6.29% | 5.27% | 7.79% | 4.13% |

These data provide further demonstrate the successful transduction of retinal photoreceptor cells and alteration of the LCA10 target position using the vectors and genome editing systems of the present disclosure.

### INCORPORATION BY REFERENCE

All references mentioned herein are hereby incorporated by reference in their entirety as if each individual reference was specifically and individually indicated to be incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

### REFERENCES

Anders et al. Nature 513(7519):569-573 (2014)
Briner et al. Mol Cell 56(2):333-339 (2014)
Cornish-Bowden Nucleic Acids Res 13(9):3021-3030 (1985)
Davis & Maizels Proc Natl Acad Sci USA 111(10):E924-E932 (2014)
Fine et al. Sci Rep 5:10777 (2015)
Frit et al. DNA Repair (Amst.) 17:81-97 (2014)
Hsu et al. Nat Biotechnol 31(9):827-832 (2013)
Iyama & Wilson DNA Repair (Amst.) 12(8):620-636 (2013)
Jiang et al. Nat Biotechnol 31(3):233-239 (2013)
Jinek et al. Science 337(6096):816-821 (2012)
Jinek et al. Science 343(6176):1247997 (2014)
Kleinstiver et al. Nature 523(7561):481-485 (2015a)
Kleinstiver et al. Nat Biotechnol 33(12):1293-1298 (2015b)
Kleinstiver et al. Nature 529(7587):490-495 (2016)
Komor et al. Nature 533(7603):420-424 (2016)
Makarova et al. Nat Rev Microbiol 9(6):467-477 (2011)
Mali et al. Science 339(6121):823-826 (2013)
Nishimasu et al. Cell 156(5):935-949 (2014)
Nishimasu et al. Cell 162(5):1113-1126 (2015)
Ran et al. Cell 154(6):1380-1389 (2013)
Tsai et al. Nat Biotechnol 34(5):483 (2016)
Zetsche et al. Nat Biotechnol 33(2):139-142 (2015)

1-141. (canceled)

142. A method of treating a subject, comprising:
contacting a retina of the subject with one or more recombinant viral vectors comprising one or more nucleic acids encoding a Cas9, a first guide RNA (gRNA) and a second gRNA,
wherein (a) the first and second gRNAs are adapted to form first and second ribonucleoprotein complexes with the Cas9, and (b) the first and second ribonucleoprotein complexes are adapted to cleave first and second cellular nucleic acid sequences on first and second sides of a CEP290 target position, thereby altering a nucleotide sequence of the CEP290 target position,
wherein the one or more recombinant viral vectors contact the retina at a dose of 1×10¹¹ viral genomes (vg)/mL to 9×10¹³ vg/mL.

143. The method of claim 142, wherein the dose is selected from the group consisting of 1×10¹¹ vg/mL, 2×10¹¹ vg/mL, 3×10¹¹ vg/mL, 4×10¹¹ vg/mL, 5×10¹¹ vg/mL, 6×10¹¹ vg/mL, 7×10¹¹ vg/mL, 8×10¹¹ vg/mL, 9×10¹¹ vg/mL, 1×10¹² vg/mL, 2×10¹² vg/mL, 3×10¹² vg/mL, 4×10¹² vg/mL, 5×10¹² vg/mL, 6×10¹² vg/mL, 7×10¹² vg/mL, 8×10¹² vg/mL, 9×10¹² vg/mL, 1×10¹³ vg/mL, 2×10¹³ vg/mL, 3×10¹³ vg/mL, 4×10¹³ vg/mL, 5×10¹³ vg/mL, 6×10¹³ vg/mL, 7×10¹³ vg/mL, 8×10¹³ vg/mL, and 9×10¹³ vg/mL.

144. The method of claim 142, wherein the subject has Leber Congenital Amaurosis-10 (LCA-10).

145. The method of claim 142, wherein the one or more recombinant viral vectors are AAV vectors.

146. The method of claim 145, wherein the AAV vectors are AAV5 vectors.

147. The method of claim 142, wherein the first cellular nucleic acid sequence is different from the second cellular nucleic acid sequence.

148. The method of claim 142, wherein the first gRNA comprises a targeting domain selected from the group consisting of SEQ ID NOS: 1-3 and the second gRNA comprises a targeting domain selected from the group consisting of SEQ ID NOS: 4-6.

149. A method of altering a retinal cell, comprising:
contacting a retina of a subject with one or more recombinant viral vectors comprising one or nucleic acid encoding a Cas9, a first gRNA and a second gRNA, wherein the first gRNA comprises a targeting domain selected from the group consisting of SEQ ID NOS: 1-3 and the second gRNA comprises a targeting domain selected from the group consisting of SEQ ID NOS: 4-6,
wherein the one or more recombinant viral vectors contact the retina at a dose of 1×10¹¹ vg/mL to 9×10¹³ vg/mL.

150. The method of claim 149, wherein the dose is selected from the group consisting of 1×10¹¹ vg/mL, 2×10¹¹ vg/mL, 3×10¹¹ vg/mL, 4×10¹¹ vg/mL, 5×10¹¹ vg/mL, 6×10¹¹ vg/mL, 7×10¹¹ vg/mL, 8×10¹¹ vg/mL, 9×10¹¹ vg/mL, 1×10¹² vg/mL, 2×10¹² vg/mL, 3×10¹² vg/mL, 4×10¹² vg/mL, 5×10¹² vg/mL, 6×10¹² vg/mL, 7×10¹² vg/mL, 8×10¹² vg/mL, 9×10¹² vg/mL, 1×10¹³ vg/mL, 2×10¹³ vg/mL, 3×10¹³ vg/mL, 4×10¹³ vg/mL, 5×10¹³ vg/mL, 6×10¹³ vg/mL, 7×10¹³ vg/mL, 8×10¹³ vg/mL, and 9×10¹³ vg/mL.

151. The method of claim 149, wherein the one or more recombinant viral vectors are administered to a subretinal space of the retina.

152. The method of claim 149, wherein the subject has Leber Congenital Amaurosis-10 (LCA-10).

153. The method of claim 149, wherein the one or more recombinant viral vectors are AAV vectors.

154. The method of claim 153, wherein the AAV vectors are AAV5 vectors.

155. A method of treating a subject having an inherited retinal dystrophy, comprising:
administering to a retina of the subject with one or more recombinant viral vectors comprising one or more nucleic acids encoding a Cas9 and a first gRNA comprising a sequence having at least 90% sequence identity to a sequence selected from SEQ ID NOS: 7 and 8;
wherein (a) the first gRNA is adapted to form a first ribonucleoprotein complex with the Cas9, and (b) the first ribonucleoprotein complex is adapted to cleave a first cellular nucleic acid sequence associated with the inherited retinal dystrophy, thereby altering the first cellular nucleic acid sequence,
wherein the one or more recombinant viral vectors are administered to the retina at a dose of 1×10¹¹ vg/mL to 9×10¹³ vg/mL.

156. The method of claim 155, wherein the dose is selected from the group consisting of 1×10¹¹ vg/mL, 2×10¹¹ vg/mL, 3×10¹¹ vg/mL, 4×10¹¹ vg/mL, 5×10¹¹ vg/mL, 6×10¹¹ vg/mL, 7×10¹¹ vg/mL, 8×10¹¹ vg/mL, 9×10¹¹ vg/mL, 1×10¹² vg/mL, 2×10¹² vg/mL, 3×10¹² vg/mL, 4×10¹² vg/mL, 5×10¹² vg/mL, 6×10¹² vg/mL, 7×10¹² vg/mL, 8×10¹² vg/mL, 9×10¹² vg/mL, 1×10¹³ vg/mL, 2×10¹³ vg/mL, 3×10¹³ vg/mL, 4×10¹³ vg/mL, 5×10¹³ vg/mL, 6×10¹³ vg/mL, 7×10¹³ vg/mL, 8×10¹³ vg/mL, and 9×10¹³ vg/mL.

157. The method of claim 155, wherein the inherited retinal dystrophy is Leber Congenital Amaurosis-10 (LCA-10).

158. The method of claim 155, wherein the one or more recombinant viral vectors are AAV vectors.

159. The method of claim 158, wherein the AAV vectors are AAV5 vectors.

160. The method of claim 155, wherein the first cellular nucleic acid sequence is different from the second cellular nucleic acid sequence.

161. The method of claim 155, wherein the first gRNA comprises a targeting domain selected from the group consisting of SEQ ID NOS: 1-3 and the second gRNA comprises a targeting domain selected from the group consisting of SEQ ID NOS: 4-6.


SEQUENCE LISTING (39 sequences)

SEQ ID NO: 1 - DNA, 22 bp, synthetic construct
  misc_feature 1..22: note = Targeting domain
  source 1..22: mol_type = other DNA
gttctgtcctcagtaaaaggta

SEQ ID NO: 2 - DNA, 20 bp, synthetic construct
  misc_feature 1..20: note = Targeting domain
  source 1..20: mol_type = other DNA
gaatagtttgttctgggtac

SEQ ID NO: 3 - DNA, 20 bp, synthetic construct
  misc_feature 1..20: note = Targeting domain
  source 1..20: mol_type = other DNA
gagaaagggatgggcactta

SEQ ID NO: 4 - DNA, 22 bp, synthetic construct
  misc_feature 1..22: note = Targeting domain
  source 1..22: mol_type = other DNA
gtcaaaagctaccggttacctg

SEQ ID NO: 5 - DNA, 20 bp, synthetic construct
  misc_feature 1..20: note = Targeting domain
  source 1..20: mol_type = other DNA
gatgcagaactagtgtagac

SEQ ID NO: 6 - DNA, 19 bp, synthetic construct
  misc_feature 1..19: note = Targeting domain
  source 1..19: mol_type = other DNA
gagtatctcctgtttggca

SEQ ID NO: 7 - DNA, 102 bp, synthetic construct
  misc_feature 1..102: note = S. aureus gRNA
  misc_difference 1..20: note = Targeting domain of 16-24 nts, each n is a, c, t, g, unknown, or other
  source 1..102: mol_type = other DNA
nnnnnnnnnnnnnnnnnnnngttttagtactctggaaacagaatctactaaaacaaggcaaaatgccgtgtttatctcgtcaacttgttggcgagatttttt

SEQ ID NO: 8 - DNA, 102 bp, synthetic construct
  misc_feature 1..102: note = S. aureus gRNA
  misc_difference 1..20: note = Targeting domain of 16-24 nts, each n is a, c, t, g, unknown, or other
  source 1..102: mol_type = other DNA
nnnnnnnnnnnnnnnnnnnngttatagtactctggaaacagaatctactataacaaggcaaaatgccgtgtttatctcgtcaacttgttggcgagatttttt

SEQ ID NO: 9 - DNA, 264 bp, synthetic construct
  misc_feature 1..264: note = U6 promoter
  source 1..264: mol_type = other DNA
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 10 - DNA, 3159 bp, synthetic construct
  misc_feature 1..3159: note = Codon optimized S. aureus Cas9
  source 1..3159: mol_type = other DNA
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 11 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 12 - DNA, 60 bp, synthetic construct
  misc_feature 1..60: note = Polyadenylation signal
  source 1..60: mol_type = other DNA
tagcaataaaggatcgtttattttcattggaagcgtgtgttggttttttgatcaggcgcg

SEQ ID NO: 13 - DNA, 617 bp, synthetic construct
  misc_feature 1..617: note = CMV promoter
  source 1..617: mol_type = other DNA
cattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgc

SEQ ID NO: 14 - DNA, 252 bp, synthetic construct
  misc_feature 1..252: note = EFS promoter
  source 1..252: mol_type = other DNA
tcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaccgcgg

SEQ ID NO: 15 - DNA, 292 bp, Homo sapiens
  source 1..292: mol_type = genomic DNA
gggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttcgcctggtgctgtgtcagccccggtctcccaggggcttcccagtggtccccaggaaccctcgacagggcccggtctctctcgtccagcaagggcagggacgggccacaggccaagggc

SEQ ID NO: 16 - DNA, 145 bp, synthetic construct
  misc_feature 1..145: note = AAV2 left ITR sequence
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 17 - DNA, 145 bp, synthetic construct
  misc_feature 1..145: note = AAV2 right ITR sequence
  source 1..145: mol_type = other DNA
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 18 - DNA, 36 bp, Mus musculus
  source 1..36: mol_type = genomic DNA
ccctcaaacacatgtctcacgcagcttagacattct

SEQ ID NO: 19 - DNA, 35 bp, Mus musculus
  source 1..35: mol_type = genomic DNA
cagaactcggtcagcatgctacagatagcttatct

SEQ ID NO: 20 - DNA, 21 bp, synthetic construct
  misc_feature 1..21: note = C1 targeting domain
  source 1..21: mol_type = other DNA
ggccggctaatttagtagaga

SEQ ID NO: 21 - DNA, 20 bp, synthetic construct
  misc_feature 1..20: note = C2 targeting domain
  source 1..20: mol_type = other DNA
gttatgaagaataatacaaa

SEQ ID NO: 22 - DNA, 5120 bp, synthetic construct
  misc_feature 1..5120: note = Reporter construct pAD26/SplitGFP+WildType/CEP290/Kan
  source 1..5120: mol_type = other DNA
acataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggaggtaagtttgtgtgattcttgaaccttgtgaaattagccatttttcttcaatatttttgtgtttggggggatttggcagattttaattaaagtttgcctgcatttatataaatttaacagagatataattatccatattattcattgtggctgaatgacttctgaatgattatctagatcattctccttaggtcacttgcatgatttagctgaatcaaacctcttttaaccagacatctaagagaaaaaggagcatgaaacaggtagaatattgtaatcaaaggagggaagcactcattaagtgcccatccctttctcttacccctgtacccagaacaaactattctcccatggtccctggcttttgttccttggaatggatgtagccaacagtagctgaaatattaagggctcttcctggaccatggatgcactctgtaaattctcatcattttttattgtagaataaatgtagaattttaatgtagaataaatttatttaatgtagaataaaaaataaaaaaactagagtagaatatcataagttacaatctgtgaatatggaccagaccctttgtagttatcttacagccacttgaactctataccttttactgaggacagaacaagctcctgatttgttcatcttcctcatcagaaatagaggcttatggattttggattattcttatctaagatcctttcacaggagtagaataagatctaattctattagctcaaaagcttttgctggctcatagagacacattcagtaaatgaaaacgttgttctgagtagctttcaggattcctactaaattatgagtcatgtttatcaatattatttagaagtaatcataatcagtttgctttctgctgcttttgccaaagagaggtgattatgttactttttatagaaaattatgcctatttagtgtggtgataatttatttttttccattctccatgtcctctgtcctatcctctccagcattagaaagtcctaggcaagagacatcttgtggataatgtatcaatgagtgatgtttaacgttatcattttcccaaagagtatttttcatctttcctaaagattttttttttttttttttgagatggagtttcattctgtcacccaggctgagtgcagtggcacgatctcggcttaacgcttactgcatcctctgcctcccagattcaagcagttctcctgcctcagcctctgagtagctgggattacaggtgtgcaccaccacaccagctaatttttttttttttttttttttttttgaggcagagtctcgctctgtcacccaggctggagtgcagtggcgccatcttggctcactgcaagctccacctcccgggttcaggccgttctcctgcctcagcctcctgagtagctggtaccacaggcacccaccatcatgcccggctaattttttgtatttttagtagagatggggtttcaccttgttagccaggatggtgtcgatctcctgaactcgtgatccacccgcctcggcctcctaaagtgctgggattacagatgtgagccaccgcacctggccccagttgtaattgtgaatatctcatacctatccctattggcagtgtcttagttttattttttattatctttattgtggcagccattattcctgtctctatctccagtcttacatcctccttactgccacaagaatgatcattctaaacatgaatcctaccctgtgactcccatgtgactccccgccttaaaaactgtcaaaagctaccggttacctgaagggtaaaagtcaagtcccctacttacctcatgtcatctagagcaagagatgaactagctgagttttctgaccacagtgttctttcttatgtatgttcttttgtacgtgctcttttctatatatagggaaccatttctctcttccagttgttttgctcagtgaatttctattcctgtttcaaaacttgttcaggcattaccttttttttcttaagcatactttttttaatggaacaaagtcactcctgtctacactagttctgcatcttatacataggttttgtacatagtacatatttatatcacatcaaattatatgtgtttacatatctgtcttccttaatggaatataagtcttttgatataaggaactatttaatttgtttctgtgtgttgagtatctcctgtttggcacagagttcaagctaatacatgagagtgattagtggtggagagccacagtgcatgtggtgtcaaatatggtgcttaggaaattattgttgctttttgagaggtaaaggttcatgagactagaggtcacgaaaatcagatttcatgtgtgaagaatggaatagatgtattatatatggaggaaattctaattttgtaaaaaactggattgtgagttttaaggagatgttatataaagttaagacaatgtcattttgtggtattggtctgaattacaatgtagtttcttagtgatatttttcctttattcagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtcgagctagcaataaaggatcgtttattttcattggaagcgtgtgttggttttttgatcaggcgcgtccaagcttgcatgctggggagagatctgcggccgctcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaccgcgggcggccgccgctagcgctaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaa

SEQ ID NO: 23 - DNA, 5120 bp, synthetic construct
  misc_feature 1..5120: note = Reporter construct pAD27/SplitGFP+Mutant/CEP290/Kan
  source 1..5120: mol_type = other DNA
acataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggaggtaagtttgtgtgattcttgaaccttgtgaaattagccatttttcttcaatatttttgtgtttggggggatttggcagattttaattaaagtttgcctgcatttatataaatttaacagagatataattatccatattattcattgtggctgaatgacttctgaatgattatctagatcattctccttaggtcacttgcatgatttagctgaatcaaacctcttttaaccagacatctaagagaaaaaggagcatgaaacaggtagaatattgtaatcaaaggagggaagcactcattaagtgcccatccctttctcttacccctgtacccagaacaaactattctcccatggtccctggcttttgttccttggaatggatgtagccaacagtagctgaaatattaagggctcttcctggaccatggatgcactctgtaaattctcatcattttttattgtagaataaatgtagaattttaatgtagaataaatttatttaatgtagaataaaaaataaaaaaactagagtagaatatcataagttacaatctgtgaatatggaccagaccctttgtagttatcttacagccacttgaactctataccttttactgaggacagaacaagctcctgatttgttcatcttcctcatcagaaatagaggcttatggattttggattattcttatctaagatcctttcacaggagtagaataagatctaattctattagctcaaaagcttttgctggctcatagagacacattcagtaaatgaaaacgttgttctgagtagctttcaggattcctactaaattatgagtcatgtttatcaatattatttagaagtaatcataatcagtttgctttctgctgcttttgccaaagagaggtgattatgttactttttatagaaaattatgcctatttagtgtggtgataatttatttttttccattctccatgtcctctgtcctatcctctccagcattagaaagtcctaggcaagagacatcttgtggataatgtatcaatgagtgatgtttaacgttatcattttcccaaagagtatttttcatctttcctaaagattttttttttttttttttgagatggagtttcattctgtcacccaggctgagtgcagtggcacgatctcggcttaacgcttactgcatcctctgcctcccagattcaagcagttctcctgcctcagcctctgagtagctgggattacaggtgtgcaccaccacaccagctaatttttttttttttttttttttttttgaggcagagtctcgctctgtcacccaggctggagtgcagtggcgccatcttggctcactgcaagctccacctcccgggttcaggccgttctcctgcctcagcctcctgagtagctggtaccacaggcacccaccatcatgcccggctaattttttgtatttttagtagagatggggtttcaccttgttagccaggatggtgtcgatctcctgaactcgtgatccacccgcctcggcctcctaaagtgctgggattacagatgtgagccaccgcacctggccccagttgtaattgtgagtatctcatacctatccctattggcagtgtcttagttttattttttattatctttattgtggcagccattattcctgtctctatctccagtcttacatcctccttactgccacaagaatgatcattctaaacatgaatcctaccctgtgactcccatgtgactccccgccttaaaaactgtcaaaagctaccggttacctgaagggtaaaagtcaagtcccctacttacctcatgtcatctaaagcaagagatgaactagctgagttttctgaccacagtgttctttcttatgtatgttcttttgtacgtgctcttttctatatatagggaaccatttctctcttccagttgttttgctcagtgaatttctattcctgtttcaaaacttgttcaggcattaccttttttttcttaagcatactttttttaatggaacaaagtcactcctgtctacactagttctgcatcttatacataggttttgtacatagtacatatttatatcacatcaaattatatgtgtttacatatctgtcttccttaatggaatataagtcttttgatataaggaactatttaatttgtttctgtgtgttgagtatctcctgtttggcacagagttcaagctaatacatgagagtgattagtggtggagagccacagtgcatgtggtgtcaaatatggtgcttaggaaattattgttgctttttgagaggtaaaggttcatgagactagaggtcacgaaaatcagatttcatgtgtgaagaatggaatagatgtattatatatggaggaaattctaattttgtaaaaaactggattgtgagttttaaggagatgttatataaagttaagacaatgtcattttgtggtattggtctgaattacaatgtagtttcttagtgatatttttcctttattcagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtcgagctagcaataaaggatcgtttattttcattggaagcgtgtgttggttttttgatcaggcgcgtccaagcttgcatgctggggagagatctgcggccgctcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaccgcgggcggccgccgctagcgctaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaa

SEQ ID NO: 24 - DNA, 5120 bp, synthetic construct
  misc_feature 1..5120: note = Reporter construct pAD28/SplitGFP+Mutant/CEP290/Inverted/Kan
  source 1..5120: mol_type = other DNA
acataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggaggtaagtttgtgtgattcttgaaccttgtgaaattagccatttttcttcaatatttttgtgtttggggggatttggcagattttaattaaagtttgcctgcatttatataaatttaacagagatataattatccatattattcattgtggctgaatgacttctgaatgattatctagatcattctccttaggtcacttgcatgatttagctgaatcaaacctcttttaaccagacatctaagagaaaaaggagcatgaaacaggtagaatattgtaatcaaaggagggaagcactcattaagtgcccatccctttctcttacccctgtacccagaacaaactattctcccatggtccctggcttttgttccttggaatggatgtagccaacagtagctgaaatattaagggctcttcctggaccatggatgcactctgtaaattctcatcattttttattgtagaataaatgtagaattttaatgtagaataaatttatttaatgtagaataaaaaataaaaaaactagagtagaatatcataagttacaatctgtgaatatggaccagaccctttgtagttatcttacagccacttgaactctatacgtaaccggtagcttttgacagtttttaaggcggggagtcacatgggagtcacagggtaggattcatgtttagaatgatcattcttgtggcagtaaggaggatgtaagactggagatagagacaggaataatggctgccacaataaagataataaaaaataaaactaagacactgccaatagggataggtatgagatactcacaattacaactggggccaggtgcggtggctcacatctgtaatcccagcactttaggaggccgaggcgggtggatcacgagttcaggagatcgacaccatcctggctaacaaggtgaaaccccatctctactaaaaatacaaaaaattagccgggcatgatggtgggtgcctgtggtaccagctactcaggaggctgaggcaggagaacggcctgaacccgggaggtggagcttgcagtgagccaagatggcgccactgcactccagcctgggtgacagagcgagactctgcctcaaaaaaaaaaaaaaaaaaaaaaaaattagctggtgtggtggtgcacacctgtaatcccagctactcagaggctgaggcaggagaactgcttgaatctgggaggcagaggatgcagtaagcgttaagccgagatcgtgccactgcactcagcctgggtgacagaatgaaactccatctcaaaaaaaaaaaaaaaaaatctttaggaaagatgaaaaatactctttgggaaaatgataacgttaaacatcactcattgatacattatccacaagatgtctcttgcctaggactttctaatgctggagaggataggacagaggacatggagaatggaaaaaaataaattatcaccacactaaataggcataattttctataaaaagtaacataatcacctctctttggcaaaagcagcagaaagcaaactgattatgattacttctaaataatattgataaacatgactcataatttagtaggaatcctgaaagctactcagaacaacgttttcatttactgaatgtgtctctatgagccagcaaaagcttttgagctaatagaattagatcttattctactcctgtgaaaggatcttagataagaataatccaaaatccataagcctctatttctgatgaggaagatgaacaaatcaggagcttgttctgtcctcagtaaaagctgaagggtaaaagtcaagtcccctacttacctcatgtcatctaaagcaagagatgaactagctgagttttctgaccacagtgttctttcttatgtatgttcttttgtacgtgctcttttctatatatagggaaccatttctctcttccagttgttttgctcagtgaatttctattcctgtttcaaaacttgttcaggcattaccttttttttcttaagcatactttttttaatggaacaaagtcactcctgtctacactagttctgcatcttatacataggttttgtacatagtacatatttatatcacatcaaattatatgtgtttacatatctgtcttccttaatggaatataagtcttttgatataaggaactatttaatttgtttctgtgtgttgagtatctcctgtttggcacagagttcaagctaatacatgagagtgattagtggtggagagccacagtgcatgtggtgtcaaatatggtgcttaggaaattattgttgctttttgagaggtaaaggttcatgagactagaggtcacgaaaatcagatttcatgtgtgaagaatggaatagatgtattatatatggaggaaattctaattttgtaaaaaactggattgtgagttttaaggagatgttatataaagttaagacaatgtcattttgtggtattggtctgaattacaatgtagtttcttagtgatatttttcctttattcagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtcgagctagcaataaaggatcgtttattttcattggaagcgtgtgttggttttttgatcaggcgcgtccaagcttgcatgctggggagagatctgcggccgctcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaccgcgggcggccgccgctagcgctaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaa

SEQ ID NO: 25 - DNA, 3944 bp, synthetic construct
  misc_feature 1..3944: note = Reporter construct pAD29/SplitGFP+Deletion/CEP290/Kan
  source 1..3944: mol_type = other DNA
acataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggaggtaagtttgtgtgattcttgaaccttgtgaaattagccatttttcttcaatatttttgtgtttggggggatttggcagattttaattaaagtttgcctgcatttatataaatttaacagagatataattatccatattattcattgtggctgaatgacttctgaatgattatctagatcattctccttaggtcacttgcatgatttagctgaatcaaacctcttttaaccagacatctaagagaaaaaggagcatgaaacaggtagaatattgtaatcaaaggagggaagcactcattaagtgcccatccctttctcttacccctgtacccagaacaaactattctcccatggtccctggcttttgttccttggaatggatgtagccaacagtagctgaaatattaagggctcttcctggaccatggatgcactctgtaaattctcatcattttttattgtagaataaatgtagaattttaatgtagaataaatttatttaatgtagaataaaaaataaaaaaactagagtagaatatcataagttacaatctgtgaatatggaccagaccctttgtagttatcttacagccacttgaactctatacctgaagggtaaaagtcaagtcccctacttacctcatgtcatctaaagcaagagatgaactagctgagttttctgaccacagtgttctttcttatgtatgttcttttgtacgtgctcttttctatatatagggaaccatttctctcttccagttgttttgctcagtgaatttctattcctgtttcaaaacttgttcaggcattaccttttttttcttaagcatactttttttaatggaacaaagtcactcctgtctacactagttctgcatcttatacataggttttgtacatagtacatatttatatcacatcaaattatatgtgtttacatatctgtcttccttaatggaatataagtcttttgatataaggaactatttaatttgtttctgtgtgttgagtatctcctgtttggcacagagttcaagctaatacatgagagtgattagtggtggagagccacagtgcatgtggtgtcaaatatggtgcttaggaaattattgttgctttttgagaggtaaaggttcatgagactagaggtcacgaaaatcagatttcatgtgtgaagaatggaatagatgtattatatatggaggaaattctaattttgtaaaaaactggattgtgagttttaaggagatgttatataaagttaagacaatgtcattttgtggtattggtctgaattacaatgtagtttcttagtgatatttttcctttattcagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtcgagctagcaataaaggatcgtttattttcattggaagcgtgtgttggttttttgatcaggcgcgtccaagcttgcatgctggggagagatctgcggccgctcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaccgcgggcggccgccgctagcgctaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaa

SEQ ID NO: 26 - RNA, 22 nt, synthetic construct
  misc_feature 1..22: note = Targeting domain
  source 1..22: mol_type = other RNA
gttctgtcctcagtaaaaggta

SEQ ID NO: 27 - RNA, 20 nt, synthetic construct
  misc_feature 1..20: note = Targeting domain
  source 1..20: mol_type = other RNA
gaatagtttgttctgggtac

SEQ ID NO: 28 - RNA, 20 nt, synthetic construct
  misc_feature 1..20: note = Targeting domain
  source 1..20: mol_type = other RNA
gagaaagggatgggcactta

SEQ ID NO: 29 - RNA, 22 nt, synthetic construct
  misc_feature 1..22: note = Targeting domain
  source 1..22: mol_type = other RNA
gtcaaaagctaccggttacctg

SEQ ID NO: 30 - RNA, 20 nt, synthetic construct
  misc_feature 1..20: note = Targeting domain
  source 1..20: mol_type = other RNA
gatgcagaactagtgtagac

SEQ ID NO: 31 - RNA, 19 nt, synthetic construct
  misc_feature 1..19: note = Targeting domain
  source 1..19: mol_type = other RNA
gagtatctcctgtttggca

SEQ ID NO: 32 - RNA, 102 nt, synthetic construct
  misc_feature 1..102: note = S. aureus gRNA
  misc_difference 1..20: note = Targeting domain of 16-24 nts, each n is a, c, u, g, unknown, or other
  source 1..102: mol_type = other RNA
nnnnnnnnnnnnnnnnnnnngttttagtactctggaaacagaatctactaaaacaaggcaaaatgccgtgtttatctcgtcaacttgttggcgagatttttt

SEQ ID NO: 33 - RNA, 102 nt, synthetic construct
  misc_feature 1..102: note = S. aureus gRNA
  misc_difference 1..20: note = Targeting domain of 16-24 nts, each n is a, c, u, g, unknown, or other
  source 1..102: mol_type = other RNA
nnnnnnnnnnnnnnnnnnnngttatagtactctggaaacagaatctactataacaaggcaaaatgccgtgtttatctcgtcaacttgttggcgagatttttt

SEQ ID NO: 34 - DNA, 36 bp, Mus musculus
  source 1..36: mol_type = genomic DNA
cagaactcggtcaggcatgctacagatagcttatct

SEQ ID NO: 35 - DNA, 31 bp, Mus musculus
  source 1..31: mol_type = genomic DNA
cagaactcggcatgctacagatagcttatct

SEQ ID NO: 36 - skipped

SEQ ID NO: 37 - DNA, 21 bp, Mus musculus
  source 1..21: mol_type = genomic DNA
catgctacagatagcttatct

SEQ ID NO: 38 - RNA, 21 nt, synthetic construct
  misc_feature 1..21: note = C1 targeting domain
  source 1..21: mol_type = other RNA
ggccggctaatttagtagaga

SEQ ID NO: 39 - RNA, 20 nt, synthetic construct
  misc_feature 1..20: note = C2 targeting domain
  source 1..20: mol_type = other RNA
gttatgaagaataatacaaa